(19) United States
(12) Patent Application Publication
(10) Pub. No.: US 2024/0293391 A1
(43) Pub. Date: Sep. 5, 2024

(54) ISOQUINOLINE AND PYRIDINE BASED CXCR4 ANTAGONISTS

(71) Applicant: Katholieke Universiteit Leuven, Leuven (BE)

(72) Inventors: Wim DEHAEN, Leuven (BE); Steven DE JONGHE, Tervuren (BE); Dominique SCHOLS, Herent (BE); Tom VAN LOY, Zoerle-Parwijs (BE)

(21) Appl. No.: 18/567,504

(22) PCT Filed: Jun. 24, 2022
(86) PCT No.: PCT/EP2022/067318

§ 371 Date: Dec. 6, 2023

(30) Foreign Application Priority Data
Jun. 24, 2021 (EP) 21181444.7
Oct. 26, 2021 (EP) 21204648.6

(51) Int. Cl. A61K 31/4725; A61K 31/47; A61K 31/4709; A61P 31/18; C07D 215/06; C07D 401/12

(52) U.S. Cl. CPC A61K 31/4725; A61K 31/47; A61K 31/4709; A61P 31/18; C07D 215/06; C07D 401/12

(57) ABSTRACT

The invention relates to isoquinoline and pyridine based CXCR4 antagonists and their use in the treatment and prevention of viral infections such as HIV.

### FIELD OF THE INVENTION

The invention relates to isoquinoline and pyridine based CXCR4 antagonists and their use in the treatment and prevention of viral infections such as HIV.

### BACKGROUND OF THE INVENTION

The CXC chemokine receptor 4 (CXCR4) is a seven transmembrane G protein-coupled receptor (GPCR), whose only endogenous ligand is the CXC chemokine ligand 12 (CXCL12), also known as stromal cell-derived factor 1 (SD33F-1). Binding of CXCL12 to CXCR4 activates different signaling pathways, leading to various biological responses such as chemotaxis, cell survival and proliferation, intracellular calcium flux and gene transcription. Under normal physiological conditions, binding of CXCL12 induces leukocytes to migrate along chemokine gradients towards sites with high concentrations of CXCL12 [Busillo & Benovic. Biochim. Biophys. Acta 2007, 1768, 952-963]. Aberrant CXCL12/CXCR4 signaling plays an important role in various pathological processes. Initially, CXCR4 was discovered as a coreceptor, in conjunction with the host CD4 receptor, for the entry of the T-tropic human immunodeficiency virus (HIV) into T-lymphocytes. CXCR4 is the main coreceptor of HIV-1 in the later stages of infection, leads to a decrease in CD4 cell count and is linked to a higher chance of advancing to the acquired immune deficiency syndrome (AIDS) [Alkhatib Curr Opin HIV AIDZ. 2009, 4, 96-103].

Mutations in the gene encoding for CXCR4 lead to truncation of its C-terminal tail and enhanced receptor activity. Clinically, it causes a rare combined immunodeficiency, characterized by warts, hypogammaglobulinemia, recurrent bacterial infection, and myelokathexis, which is known as the WHIM syndrome [Heusinkveld et al. J. Clin. Immunol. 2019, 1990, 532-556]. Overexpression of CXCR4 is reported for more than 20 types of solid and hematological cancers and is correlated with poor prognosis. CXCR4 was shown to be essential for various fundamental aspects of cancer, such as primary tumor growth, cancer cell migration, and the establishment of metastatic sites. This points towards CXCR4 as a general driver of human malignancies [Luker et al. J. Leukoc. Biol. 2021, 109, 969-989]. CXCR4 has also been implicated in various inflammatory disorders, such as rheumatoid arthritis, inflammatory bowel disease and asthma [García-cuesta et al. Front. Endocrinol. 2019, 10, 1-16].

The promise of CXCR4 as drug target spurred the search for small molecule CXCR4 antagonists [Debnath et al. Theranostics 2013, 3, 47-75; Tahirovic et al. Expert Opin. Ther. Pat 2020, 30, 87-101]. AMD3100 (Plerixafor, compound 1, FIG. 1) received marketing approval for hematopoietic stem cell mobilization for transplantations in case of non-Hodgkin lymphoma and multiple myeloma [Smith et al. Cancer Res. 2004, 64, 8604-8612]. In addition, AMD3100 has been used as chemical tool to demonstrate that small molecule antagonism of CXCR4 is a promising strategy for the treatment of different cancers, such as breast cancer [Smith et al. Cancer Res. 2004, 64, 8604-8612], prostate cancer [Zhu et al. J. Cell. Physiol. 2019, 234, 11746-11759] and ovarian cancer [Alsina-sanchís et al. Mol. Cancer Ther. 2018, 17, 532-543]. The lack of oral bioavailability of AMD3100 prompted the search for orally bioavailable small molecules. Different scaffolds have been elaborated, but most studies dealt with the tetrahydroquinoline based derivatives. Among this class of compounds, AMD11070 (mavorixafor, compound 2, FIG. 1) is noteworthy. This analogue has been clinically evaluated as an antiretroviral agent, but was then halted for further development as anti-HIV drug [Mosi et al. Biochem. Pharmacol. 2012, 83, 472-479]. Recently, a phase 3 clinical trial for the treatment of the WHIM syndrome was initiated. Since then, many structural modifications have been pursued within this series. In most of these efforts, the 8-aminotetrahydroquinoline head piece was kept intact and structural variation was pursued on the lower part of the molecule. The basic n-butylamine side chain was removed and a piperazine ring was appended to the lower part, giving rise to GSK-812397 (compound 3, FIG. 1) [Jenkinson et al. Antimicrob. Agents Chemother. 2010, 54, 817-824]. It has been demonstrated that the benzimidazole moiety can be replaced by various mono- and bicyclic heteroaromatics, as exemplified by the isoquinoline congener 4 [Miller et al. Bioorg. Med. Chem. Lett. 2010, 20, 3026-3030]. Substitution of the bicyclic heteroaromatic moiety by a partially saturated ring system yielded the tetrahydroisoquinoline derivative 5 (also known as TIQ15, FIG. 1), which is a highly potent and selective CXCR4 antagonist [Truax et al. ACS Med. Chem. Lett. 2013, 4, 1025-1030]. Further optimization yielded compound 6, which has improved in vitro ADME properties compared to compound 5 [Nguyen et al. Med. Chem. 2018, 61, 7168-7188]. In contrast to the lower part, the upper tetrahydroquinoline moiety of AMD-11070 has been left relatively unexplored. It was shown that the stereochemistry of the tetrahydroquinoline moiety plays an important role in CXCR4 antagonism, with the (S)-enantiomer being more active than the (R)-enantiomer [Gudmundsson et al. Bioorg. Med. Chem. Lett. 2009, 19, 5048-5052]. Structural simplification of the tetrahydroquinoline moiety afforded a series of 2-(aminomethyl)pyridine analogues, from which compound 7 (FIG. 1) is a representative example. This compound displayed excellent CXCR4 antagonism and showed an improved metabolic stability (when compared to the corresponding tetrahydroquinoline congeners) [Wilson et al. ACS Med. Chem. Lett. 2018, 9, 17-22]. Recently, it was shown that aromatization of the tetrahydroquinoline core yielding the aminoquinoline derivative 8 that displayed very potent CXCR4 affinity [Lin et al. Bioorg. Chem. 2020, 99, 103824.]. A major advantage of the open scaffolds and the fully aromatic compounds are the lack of chirality.

### SUMMARY OF THE INVENTION

The invention is summarized in the following statements:
1. A compound with formula I

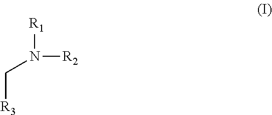

wherein R₁ is

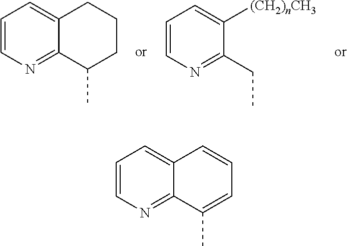

With n=0-5
wherein R₂ is —(CH₂)ₘ—NH₂ with m=1-5, and
wherein R³ is

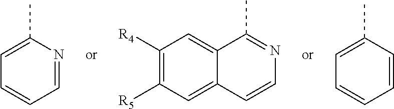

wherein R₄ and R₅ are independently selected from H, OH, CH₃, OCH₃, OCH₂CH₃ and halogen.
2. The compound according to claim 1
   wherein R₁ is

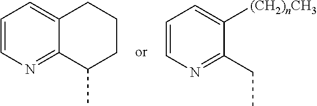

With n=0-5
wherein R₂ is —(CH₂)ₘ—NH₂ with m=1-5, and
wherein R³ is

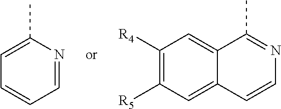

wherein R₄ and R₅ are independently selected from H, OH, CH₃, OCH₃, OCH₂CH₃ and halogen.
3. The compound according to statement 1 or 2, wherein m=4.
4. The compound according to any one of statements 1 to 3, wherein R₄ is hydrogen and R₅ is an halogen.
5. The compound according to any one of statements 1 to 4, wherein the halogen is Br, F or I.
6. The compound according to any one of statements 1 to 5, wherein n in R₁ is 1.
7. The compound according to any one of statements 1 to 6, wherein R₄ and/or R₅ is OCH₃.
8. The compound according to any one of statements 1 to 7, for use a medicament.
9. A pharmaceutical composition comprising a compound according to any one of statement 1 to 7, and a pharmaceutically acceptable carrier.
10. The compound according to any one of statements 1 to 7 for use in the treatment or prevention of a viral infection. The viral infection can be a retroviral or lentiviral infection.
11. The compound according to any one of statements 1 to 7 for use in the treatment or prevention of an HIV infection.
12. A method of treatment or prevention of a viral infection comprising the step of administering a compound according to any one of claims 1 to 7.
13. The method according to claim 12, wherein the viral infection is a retroviral or lentiviral infection.
14. The method according to claim 12 or 13, wherein the viral infection is a retroviral or lentiviral infection.

### DESCRIPTION OF THE INVENTION

### Figure Legends

FIG. 1. Small molecule CXCR4 antagonists

FIG. 2. CXCR4 antagonists of the present invention

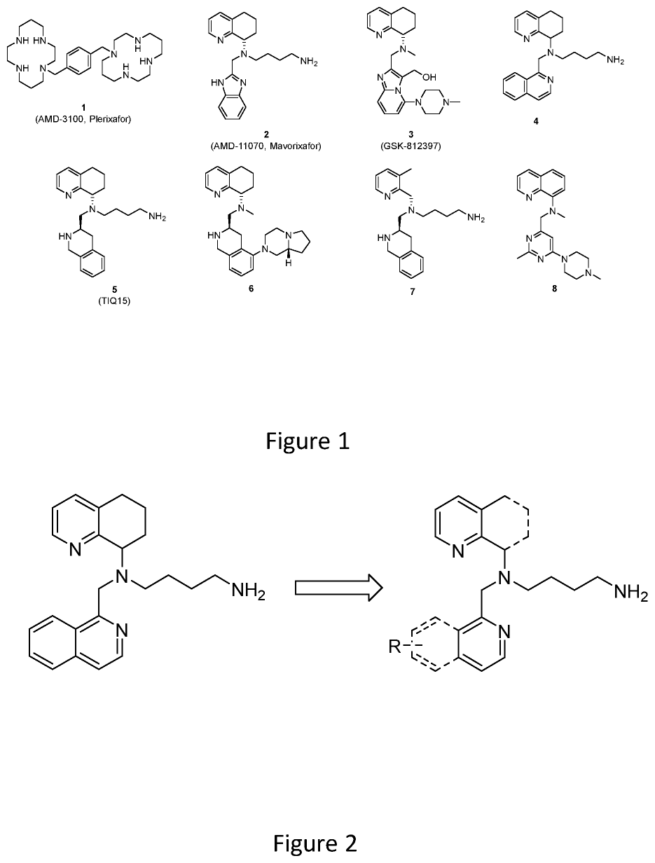

In 2016, a metal-free three-component reaction was published for the synthesis of 1,5-disubstituted [Thomas et al. Chem. Commun. 2016, 52, 2885-2888; Thomas et al. Chem. Commun. 2016, 52, 9236-9239; Parkash et al. Chem. Rec. 2021, 21, 376-385] 1,2,3-triazoles, known as the ‘triazolization reaction of ketones’. Acid-mediated denitrogenative ring opening of triazoloisoquinolines furnished various 1-methyleneisoquinolines [23]. Here, these synthetic methodologies were applied for the synthesis of a series of novel isoquinoline based CXCR4 antagonists, derived from lead compound 4. To probe the optimal substitution pattern for CXCR4 antagonism, various small substituents (R=F, Br, OCH₃) were introduced on the isoquinoline moiety. In order to delineate if CXCR4 antagonism depends on the presence of a bicyclic isoquinoline moiety, the corresponding pyridine congeners were also prepared. As head groups, the classical 8-aminotetrahydroquinoline moiety, as well as the simplified 3-methylpyridinyl group, were selected (FIG. 2).

### Chemistry

The synthesis of the tetrahydroquinoline derivatives is shown in Scheme 1. A multi-component reaction between acetophenone derivatives 9a-d, 2,2-dimethoxyethan-1-amine 10 and 4-nitrophenyl azide (4-NPA) 11 in toluene at 100° C. yielded the dimethyl acetal-substituted triazoles 12a-d. Conversion into the triazolo[5,1-a]isoquinolines 13a-d was achieved using concentrated sulfuric acid in a modified Pomeranz-Fritsch reaction [Opsomer et al. Org. Lett. 2020, 22, 3596-3600]. This was followed by an acid-catalyzed ring opening of the triazole moiety [Jones et al. J. Chem. Soc., Perkin Trans. 1 1985, 19, 2719-2723] using water as the nucleophile to afford the 1-hydroxymethylisoquinolines 14a-d. Upon treatment with SOCl₂ at room temperature, the desired 1-chloromethyl isoquinoline derivatives 15a-d were obtained.

The selective mono-protection [Gerber et al. Tetrahedron 2017, 73, 6347-6355; Manickam et al. Bioorg. Med. Chem. Lett. 2021, 31, 127685] of butane-1,4-diamine 16 with di-tert-butyl dicarbonate afforded intermediate 17, which was used for further reaction without any purification. Reductive amination with 6,7-dihydroquinolin-8(5H)-one in the presence of NaBH(OAc)₃ at room temperature, yielded compound 18 in 73% yield. The subsequent alkylation of amine 18 by treatment with 1-chloromethylisoquinolines 15a-e in the presence of K₂CO₃ under reflux temperature was followed by acidic cleavage of the Boc protecting group, yielding final compounds 19a-e. A reductive amination between amine 18 and the commercially available picolinaldehyde in presence of NaBH(OAc)₃, followed by Boc deprotection, furnished final compound 20 [Miller et al. cited above; Li et al. Eur. J. Med. Chem. 2018, 149, 30-44].

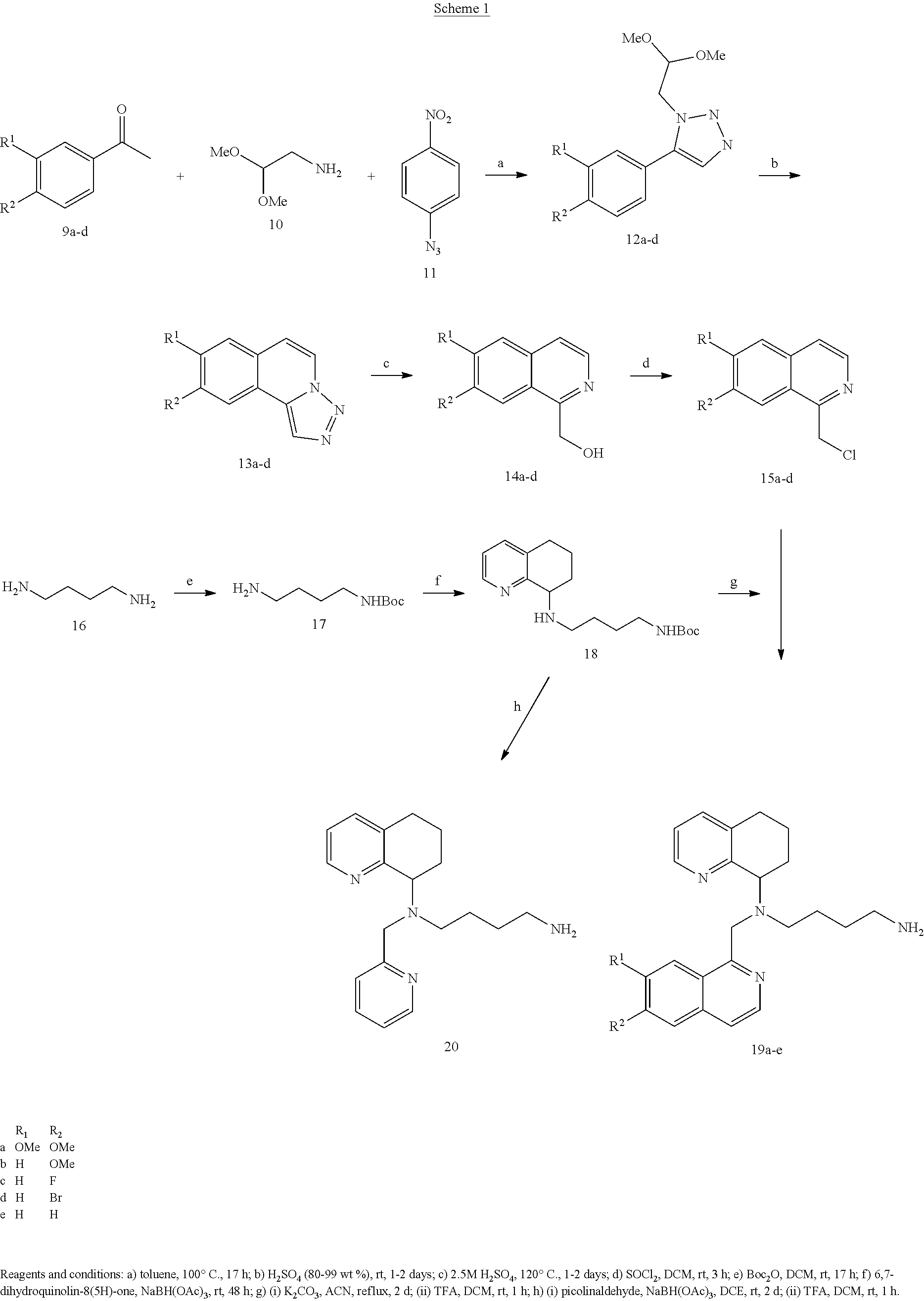

Reagents and conditions: a) toluene, 100° C., 17 h; b) H₂SO₄ (80-99 wt %), rt, 1-2 days; c) 2.5M H₂SO₄, 120° C., 1-2 days; d) SOCl₂, DCM, rt, 3 h; e) Boc₂O, DCM, rt, 17 h; f) 6,7-dihydroquinolin-8(5H)-one, NaBH(OAc)₃, rt, 48 h; g) (i) K₂CO₃, ACN, reflux, 2 d; (ii) TFA, DCM, rt, 1 h; h) (i) picolinaldehyde, NaBH(OAc)₃, DCE, rt, 2 d; (ii) TFA, DCM, rt, 1 h.

The synthesis of the 3-methylpyridine derivatives is depicted in Scheme 2. Formylation of the lithium salt of 2-bromo-3-methylpyridine 21 with DMF afforded 3-methylpicolinaldehyde 22 [28], which was then used in a reductive amination with amine 17, yielding key intermediate 23. Alkylation of 23 with 1-chloromethylisoquinolines 15a-e or a reductive amination with picolinaldehyde yielded target compounds 24a-e and 25, respectively [Eun et al. Arch. Pharmacal Res. 2009, 32, 1673-1679].

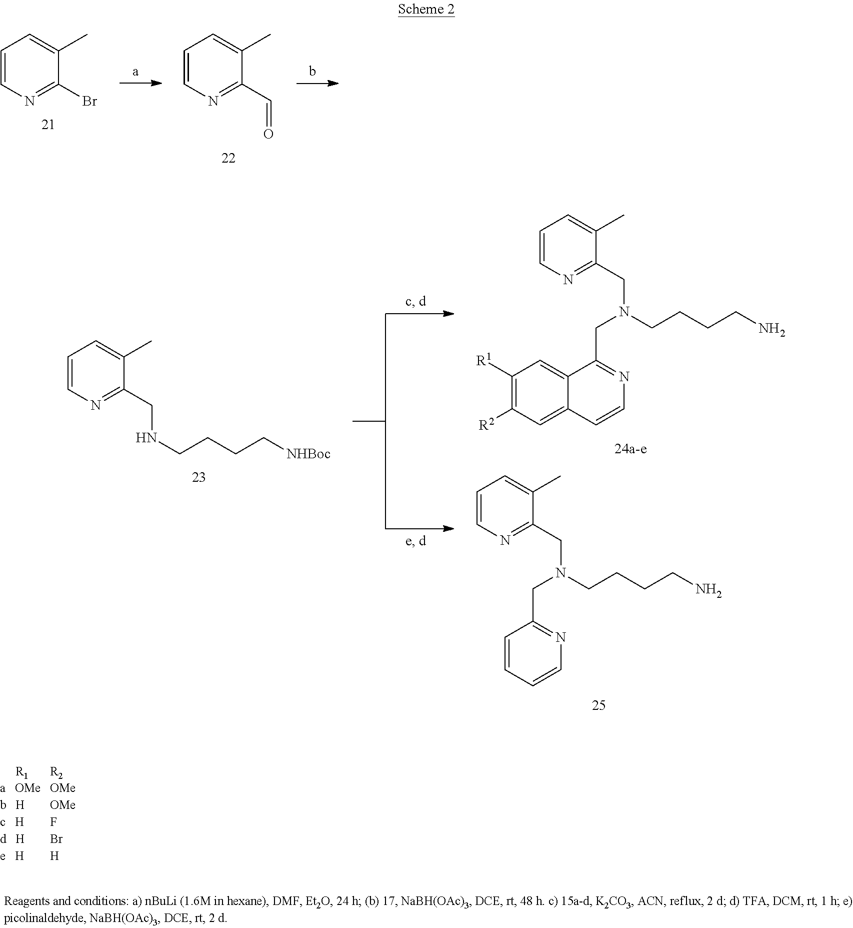

Reagents and conditions: a) nBuLi (1.6M in hexane), DMF, Et₂O, 24 h; (b) 17, NaBH(OAc)₃, DCE, rt, 48 h. c) 15a-d, K₂CO₃, ACN, reflux, 2 d; d) TFA, DCM, rt, 1 h; e) picolinaldehyde, NaBH(OAc)₃, DCE, rt, 2 d.

The synthesis of the quinoline derivatives is shown in Scheme 3. The amino group of commercially available 4-aminobutan-1-ol 26 was reacted with phthalic anhydride yielding intermediate 27 in high yield. The next step involves a Swern oxidation to convert the primary alcohol to the corresponding aldehyde, affording compound 28. Reductive amination with 8-aminoquinoline, using sodium triacetoxyborohydride and acetic acid, furnished compound 29. Alkylation of the exocyclic amino group with various benzylhalides yielded compounds 30a-d. Finally, hydrazine mediated deprotection of the phthalic protecting group afforded the target compounds 31a-d.

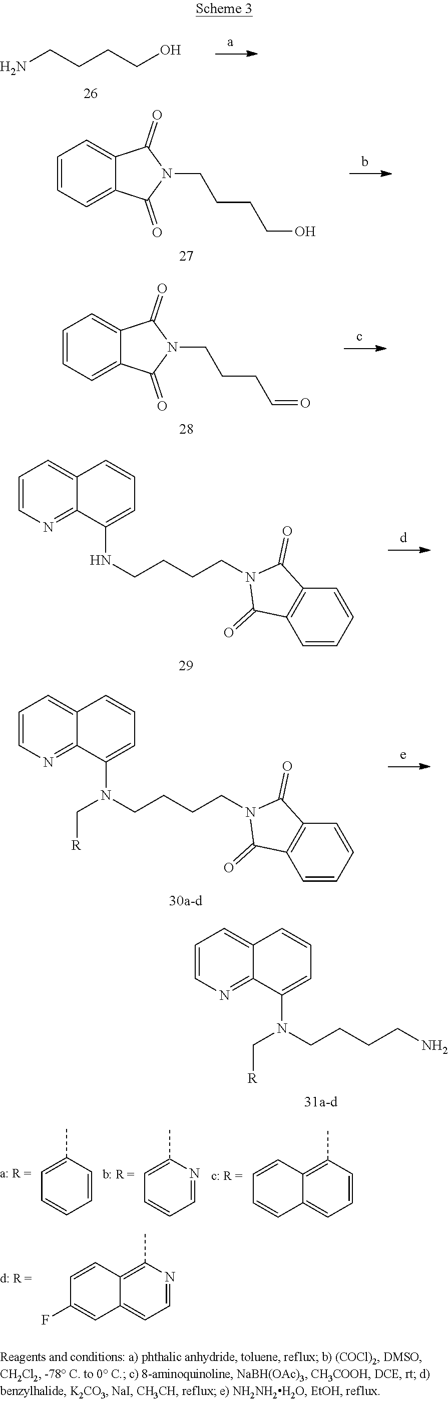

Reagents and conditions: a) phthalic anhydride, toluene, reflux; b) (COCl)₂, DMSO, CH₂Cl₂, −78° C. to 0° C.; c) 8-aminoquinoline, NaBH(OAc)₃, CH₃COOH, DCE, rt; d) benzylhalide, K₂CO₃, NaI, CH₃CN, reflux; e) NH₂NH₂·H₂O, EtOH, reflux.

### Biological Evaluation

Compounds 19a-e, 20, 24a-e and 25 were evaluated in a panel of in vitro cell-based assays (Table 1) [Hout et al. PLOS One 2017, 12, 1-18]. First, the ability of the compounds to compete with fluorescently labeled CXCL12 (CXCL12^{AF647}) for binding at CXCR4 was determined. Secondly, since binding of CXCL12 to CXCR4 results into a transient increase of cytosolic calcium levels, the inhibition of this CXCL12-induced calcium mobilization by the various compounds was also investigated. Finally, given the role of CXCR4 as a major coreceptor for HIV entry, the antiviral activity of the compounds against the X4-tropic wild type HIV-1 NL4-3 strain and the HIV-2 ROD strain was determined in MT-4 cells. In parallel, the cytotoxicity of the compounds for uninfected MT-4 cells was assessed. In each of these assays, plerixafor was included as positive control and showed potent inhibitory activity in the various assays.

All newly synthesized isoquinoline based derivatives were able to compete with CXCL12 for binding to CXCR4, as evidenced by IC₅₀ values of less than 40 nM for all analogues, the only exception being compound 24b that displayed an IC₅₀ value of 0.89 μM in the binding assay. It is noteworthy that a structurally simplified analogue 25 carrying a 3-methylpyridinyl moiety (instead of the THQ group) and a pyridinyl ring (instead of the isoquinoline ring) is still endowed with very potent CXCR4 binding affinity (IC₅₀=0.6 nM), making this a very ligand-efficient molecule. A similar trend was found in the CXCR4 calcium mobilization assay: all compounds were active (IC₅₀ values of less than 1 μM), with compound 24b being the least active (IC₅₀=3.85 μM). However, all compounds do show a diminished potency in the calcium flux assay, when compared to the binding assay, which is also observed for plerixafor. The HIV screening revealed a number of analogues (compounds 19a-c, 19e, 20, 24a and 24c) that show excellent antiviral activity against HIV-1 and HIV-2 (EC₅₀<100 nM) and at the same time, lack cytotoxicity for the MT4 cell line. Compounds that were less active in the calcium mobilization assay (such as compounds 19b, 24b, 24 d-e and 25) also displayed a diminished activity as anti-HIV agent. Overall, the biological profile of compound 24c looks very promising, since it shows low nM activity in all assays (IC₅₀ values between 0.6 and 6 nM) and lacks cytotoxicity (CC₅₀=31 μM).

TABLE 1

| | | | HIV | | |
|---|---|---|---|---|---|
| | CXCR4 | | HIV-1 | HIV-2 | |
| | Binding | Calcium | NL4.3 | ROD | |
| | IC₅₀ | IC₅₀ | EC₅₀ | EC₅₀ | CC₅₀ |
| Cmpd | (μM)^{a} | (μM)^{b} | (μM)^{c} | (μM)^{c} | (μM)^{d} |
| 19a | 0.0072 | 0.025 | 0.074 | 0.042 | >100 |
| 19b | 0.00025 | 0.028 | 0.061 | 0.077 | 11.11 |
| 19c | 0.0089 | 0.033 | 0.016 | 0.011 | 18.12 |
| 19d | 0.026 | 0.44 | 0.55 | 0.55 | 43.58 |
| 19e | 0.0092 | 0.13 | 0.084 | 0.026 | 26.7 |
| 20 | 0.0026 | 0.057 | 0.058 | 0.058 | 55.45 |
| 24a | 0.0032 | 0.013 | 0.023 | 0.024 | >100 |
| 24b | 0.89 | 3.85 | >9.27 | >9.27 | 9.27 |
| 24c | 0.00064 | 0.0061 | 0.0062 | 0.0038 | 30.77 |
| 24d | 0.039 | 0.69 | 2.54 | 2.54 | 29.48 |
| 24e | 0.026 | 0.93 | 0.77 | 2.29 | 40.89 |
| 25 | 0.0062 | 0.18 | 0.23 | 0.17 | 79.85 |
| 31a | >100 | >50 | >41.57 | >41.57 | 41.57 |
| 31b | 0.036 | 5.5 | 0.38 | >9.46 | 9.46 |
| 31c | 0.0023 | 0.005 | 0.018 | 0.0013 | 2.98 |
| Plerixafor | 0.025 | 0.26 | 0.0049 | 0.0081 | 6.29 |

Values are the mean of at least two independent experiments
^{a}Concentration needed to inhibit CXCL12 receptor binding by 50%
^{b}Concentration needed to inhibit CXCL12-induced calcium signaling by 50%
^{c}Concentration required to achieve 50% protection of MT-4 cells against HIV-induced cytopathicity
^{d}Concentration required to reduce the viability of mock-infected MT4 cells by 50%

### EXAMPLES

### General Information

All chemicals were purchased from Acros Organics, Merck, Alfa Aesar, Fluorochem or TCI Europe and were used as received. For column chromatography, 70-230 mesh silica 60 (Acros) was used as the stationary phase. NMR spectra were recorded on commercial instruments (Bruker Avance 300 or Bruker Avance III HD 400 or Bruker Avance II+ 600) and chemical shifts (δ) are reported in parts per million (ppm) referenced to tetramethylsilane (¹H), or the internal (NMR) solvent signal (¹³C). High-resolution mass spectra were acquired on a quadrupole orthogonal acceleration time-of-flight mass spectrometer (Synapt G2 HDMS, Waters, Milford, MA). Samples were infused at 3 μL/min and spectra were obtained in positive ionization mode with a resolution of 15000 (FWHM) using leucine enkephalin as lock mass. Melting points were determined using a Reichert Thermovar apparatus. Recombinant human CXCL12 was purchased from Peprotech and human CXCL12-Alexa 647® (CXCL12AF647) was obtained from Almac.

### Synthesis of Dimethyl Acetal-Substituted Triazoles (12a-d)

### General Procedure

Commercially available substituted acetophenones 9a-d (1 eq., 2.5 mmol) and 2-aminoacetaldehyde dimethyl acetal 10 (1.1 eq., 2.75 mmol, 289 mg) were added to an oven-dried screw-capped reaction tube equipped with a magnetic stirring bar. The mixture was dissolved in dry toluene (2 ml) followed by addition of 4-nitrophenyl azide 11 (1.5 eq., 3.75 mmol, 615 mg) and stirred overnight at 100° C. in an aluminum heating block. The crude residue was purified via silica gel flash chromatography, yielding the title compounds. The following compounds were made according to this general procedure.

### 1-(2,2-Dimethoxyethyl)-5-(3,4-dimethoxyphenyl)-1H-1,2,3-triazole (12a)

This compound was prepared from 3′,4′-dimethoxyacetophenone 9a (415.45 mg, 2.50 mmol). The crude residue was purified by silica gel flash chromatography using DCM/EtOAc (95:5) as mobile phase, yielding the title compound as a yellowish-orange solid (654 mg, 89%); mp 90-92° C. ¹H NMR (400 MHZ, CDCl₃) δ: 7.67 (s, 1H), 7.10 (d, J=2.0 Hz, 1H), 7.06 (dd, J=8.2, 2.0 Hz, 1H), 6.97 (d, J=8.2 Hz, 1H), 4.93 (t, J=5.6 Hz, 1H), 4.41 (d, J=5.7 Hz, 2H), 3.94 (s, 3H), 3.92 (s, 3H), 3.37 (s, 6H). ¹³C NMR (101 MHZ, CDCl₃) δ: 150.0, 149.3, 139.0, 132.6, 122.1, 119.2, 112.4, 111.5, 103.7, 56.1, 56.1, 55.4, 49.7. HRMS (ESI-Q-TOF): m/z [M+H]⁺ calcd for C₁₄H₁₉N₃O₄: 294.1448; found: 294.1455

### 1-(2,2-Dimethoxyethyl)-5-(4-methoxyphenyl)-1H-1,2,3-triazole (12b)

This compound was prepared from 4′-methoxyacetophenone 9b (375.45 mg, 2.50 mmol). The crude residue was purified by silica gel flash chromatography using DCM/EtOAc (95:5) as mobile phase, yielding the title compound as a yellowish-orange viscous oil (488 mg, 74%). ¹H NMR (400 MHZ, CDCl₃) δ: 7.64 (s, 1H), 7.42 (d, J=8.7 Hz, 2H), 7.01 (d, J=8.7 Hz, 2H), 4.88 (t, J=5.6 Hz, 1H), 4.39 (d, J=5.7 Hz, 2H), 3.85 (s, 3H), 3.33 (s, 6H). ¹³C NMR (101 MHZ, CDCl₃) δ: 160.4, 138.7, 132.5, 130.5, 118.9, 114.4, 103.3, 55.3, 55.1, 49.4. HRMS (ESI-Q-TOF): m/z [M+H]⁺ calcd for C₁₃H₁₇N₃O₃: 264.1343; found: 264.1346.

### 1-(2,2-Dimethoxyethyl)-5-(4-fluorophenyl)-1H-1,2,3-triazole (12c)

This compound was prepared from 4′-fluoroacetophenone 9c (345.35 mg, 2.50 mmol). The crude residue was purified by silica gel flash chromatography using DCM/EtOAc (95:5) as mobile phase, yielding the title compound as a reddish viscous oil (447 mg, 71%). ¹H NMR (400 MHZ, CDCl₃) δ: 7.65 (s, 1H), 7.47 (dd, J=8.8, 5.2 Hz, 2H), 7.16 (t, J=8.6 Hz, 2H), 4.86 (t, J=5.6 Hz, 1H), 4.35 (d, J=5.6 Hz, 2H), 3.33 (s, 6H). ¹³C NMR (101 MHZ, CDCl₃) δ: 164.6, 162.1, 138.0, 132.9, 131.2, 122.9, 116.2, 103.6, 55.5, 49.6. HRMS (ESI-Q-TOF): m/z [M+H]⁺ calcd for C₁₂H₁₄FN₃O₂: 252.1143; found: 252.1139.

### 5-(4-Bromophenyl)-1-(2,2-dimethoxyethyl)-1H-1,2,3-triazole (12 d)

This compound was prepared from 4′-bromoacetophenone 9 d (497.6 mg, 2.50 mmol). The crude residue was purified by silica gel flash chromatography using DCM/EtOAc (95:5) as mobile phase, yielding the title compound as an orange viscous oil (485 mg, 62%). ¹H NMR (400 MHZ, CDCl₃) δ: 7.68 (s, 1H), 7.62 (d, J=8.5 Hz, 2H), 7.37 (d, J=8.5 Hz, 2H), 4.87 (t, J=5.6 Hz, 1H), 4.36 (d, J=5.6 Hz, 2H), 3.34 (s, 6H). ¹³C NMR (101 MHZ, CDCl₃) δ: 138.1, 133, 132.4, 130.9, 125.9, 124.2, 103.7, 55.6, 49.8. HRMS (ESI-Q-TOF): m/z [M+H]⁺ calcd for C₁₂H₁₄BrN₃O₂: 312.0343; found: 312.0338.

### Synthesis of 1,2,3-triazolo[5,1-a]isoquinolines (13a-d)

### General Procedure

To an open round-bottom flask equipped with a magnetic stirring bar containing the triazole 12a-d (1.2 mmol) was added concentrated H₂SO₄ (80-95%, 2.5-4 mL), while stirring at 0° C. in an ice bath. After cooling for 30 minutes, the reaction mixture was stirred overnight at room temperature. The reaction mixture was poured over ice and neutralized by slowly adding a 3 M aqueous NaOH solution. The aqueous phase was subsequently extracted five times with dichloromethane. The combined organic layers were dried over MgSO₄ and concentrated in vacuo. The crude residue was purified by flash chromatography, yielding the title compound. The following compounds were made according to this procedure.

### 8,9-Dimethoxy-[1,2,3]triazolo[5,1-a]isoquinoline (13a)

This compound was prepared from triazole 12a (354 mg, 1.21 mmol). The crude residue was purified by silica gel flash chromatography using DCM/EtOAc (80:20) as mobile phase, yielding the title compound as an off-white solid (274 mg, 99%). mp 221-224° C. ¹H NMR (400 MHZ, CDCl₃) δ: 8.42 (d, J=7.3 Hz, 1H), 8.29 (s, 1H), 7.39 (s, 1H), 7.13 (s, 1H), 7.07 (d, J=7.3 Hz, 1H), 4.05 (s, 3H), 4.02 (s, 3H). ¹³C NMR (101 MHZ, CDCl₃) δ: 150.7, 150.6, 132.3, 124.5, 123.9, 121.0, 117.3, 115.4, 107.9, 104.5, 56.3, 56.2. HRMS (ESI-Q-TOF): m/z [M+H]⁺ calcd for C₁₂H₁₁N₃O₂: 230.0924; found: 230.0936.

### 8-Methoxy-[1,2,3]triazolo[5,1-a]isoquinoline (13b)

This compound was prepared from triazole 12b (301 mg, 1.14 mmol). The crude residue was purified by silica gel flash chromatography using DCM/EtOAc (80:20) as mobile phase, yielding the title compound as an off-white solid (226 mg, 99%). mp 133-134° C. ¹H NMR (400 MHZ, CDCl₃) δ: 8.43 (d, J=7.4 Hz, 1H), 8.26 (s, 1H), 7.96 (d, J=8.8 Hz, 1H), 7.20 (dd, J=8.8, 2.6 Hz, 1H), 7.12 (d, J=2.5 Hz, 1H), 7.06 (d, J=7.4 Hz, 1H), 3.92 (s, 3H). ¹³C NMR (101 MHZ, CDCl₃) δ: 160.0, 132.5, 130.7, 125.5, 124.6, 122.8, 118.3, 116.7, 115.6, 108.8, 55.5. HRMS (ESI-Q-TOF): m/z [M+H]⁺ calcd for C₁₁H₉N₃O: 200.0818; found: 200.0826

### 8-Fluoro-[1,2,3]triazolo[5,1-a]isoquinoline (13c)

This compound was prepared from triazole 12c (301 mg, 1.2 mmol) and H₂SO₄ (80%, aq.). The crude residue was purified by silica gel flash chromatography using DCM/EtOAc (80:20) as mobile phase, yielding the title compound as an off-white solid (196 mg, 87%). mp 123-126° C. ¹H NMR (400 MHZ, CDCl₃) δ: 8.55 (d, J=7.4 Hz, 1H), 8.38 (s, 1H), 8.15 (dd, J=8.6, 5.3 Hz, 1H), 7.47 (d, J=9 Hz, 1H), 7.4 (d, J=8.4 Hz, 1H), 7.15 (d, J=7.4 Hz, 1H). ¹³C NMR (101 MHZ, CDCl₃) δ 162.71 (d, J=250.3 Hz), 138.48, 133.55, 130.96 (d, J=8.5 Hz), 126.62 (d, J=9.1 Hz), 125.63, 123.90, 117.79 (d, J=23.9 Hz), 115.42 (d, J=3.4 Hz), 113.05 (d, J=22.2 Hz). HRMS (ESI-Q-TOF): m/z [M+H]⁺ calcd for C₁₀H₆FN₃: 188.0618; found: 188.0617.

### 8-Bromo-[1,2,3]triazolo[5,1-a]isoquinoline (13 d)

This compound was prepared from triazole 12 d (375 mg, 1.2 mmol). The crude residue was purified by silica gel flash chromatography using DCM/EtOAc (80:20) as mobile phase, yielding the title compound as an off-white solid (285 mg, 95%). mp 209-211° C. ¹H NMR (400 MHZ, CDCl₃) δ: 8.54 (d, J=7.4 Hz, 1H), 8.42 (s, 1H), 8.02 (d, J=8.5 Hz, 1H), 7.97 (d, J=1.9 Hz, 1H), 7.78 (dd, J=8.5, 1.9 Hz, 1H), 7.3 (d, J=7.4 Hz, 1H). ¹³C NMR (101 MHZ, CDCl₃) δ: 131.8, 131.7, 130.3, 129.8, 125.7, 125.3, 123.5, 1229, 121.4, 114.6. HRMS (ESI-Q-TOF): m/z [M+H]⁺ calcd for C₁₀H₆BrN₃: 247.9818; found: 247.9812.

### Synthesis of 1-hydroxymethylisoquinolines (14a-d)

### General Procedure

A solution of the triazoloisoquinoline 13a-d (1.2 mmol) in a 2.5M H₂SO₄ solution (8-10 ml) was heated at 120° C. until completion of the reaction (24-36 hours). Subsequently, the reaction mixture was poured over ice and neutralized by adding a saturated aqueous NaHCO₃ solution. The aqueous phase was extracted three times with dichloromethane. The combined organic phases were dried over MgSO₄ and concentrated in vacuo to afford the title compound, which was used for the next step without further purification. The following compounds were made according to this procedure.

### (6,7-Dimethoxyisoquinolin-1-yl)methanol (14a)

This compound was prepared from triazoloisoquinoline 13a (275 mg, 1.2 mmol). The title compound was isolated as an off-white solid (242 mg, 92%). mp 131-133° C. ¹H NMR (400 MHZ, CDCl₃) δ: 8.33 (d, J=5.7, 1H), 7.46 (d, J=5.6, 1H), 7.11 (s, 1H), 7.05 (s, 1H), 5.13 (s, 2H), 4.04 (s, 3H), 4.03 (s, 3H). ¹³C NMR (101 MHZ, CDCl₃) δ: 155.5, 153.7, 151.1, 140.1, 133.5, 119.9, 106.1, 101.9, 62.1, 56.8. HRMS (ESI-Q-TOF): m/z [M+H]⁺ calcd for C₁₂H₁₃NO₃: 220.0968; found: 220.0974.

### (6-Methoxyisoquinolin-1-yl)methanol (14b)

This compound was prepared from triazoloisoquinoline 13b (240 mg, 1.2 mmol). The title compound was isolated as an off-white solid (173 mg, 76%); mp 63-65° C. 1H NMR (400 MHZ, CDCl₃) δ: 8.30 (d, J=5.8 Hz, 1H), 7.76 (d, J=9.2 Hz, 1H), 7.43 (d, J=5.7 Hz, 1H), 7.19 (s, 1H), 7.04 (s, 1H), 5.11 (s, 2H), 3.88 (s, 3H). ¹³C NMR (101 MHz, CDCl₃) δ: 160.7, 156.5, 140.5, 137.9, 124.8, 120.2, 120.1, 119.5, 104.8, 60.9, 55.2. HRMS (ESI-Q-TOF): m/z [M+H]⁺ calcd for C₁₁H₁NO₂: 190.0862; found: 190.0869.

### (6-Fluoroisoquinolin-1-yl)methanol (14c)

This compound was prepared from triazoloisoquinoline 13c (225 mg, 1.2 mmol). The title compound was isolated as an off-white solid (194 mg, 91%). mp 72-74° C. 1H NMR (400 MHZ, CDCl₃) δ: 8.45 (d, J=5.8 Hz, 1H), 7.96 (dd, J=9.1, 5.3 Hz, 1H), 7.57 (d, J=5.8 Hz, 1H), 7.48 (dd, J=9.2, 2.5 Hz, 1H), 7.3 (td, J=8.5, 2.5 Hz, 1H), 5.22 (s, 2H), 4.93 (s, 1H). ¹³C NMR (101 MHZ, CDCl₃) δ: 163.9, 161.4, 156.9, 140.8, 137.1, 125.8, 121.6, 119.4, 117.4, 110.3. HRMS (ESI-Q-TOF): m/z [M+H]⁺ calcd for C₁₀H₈FNO: 178.0663; found: 178.0659.

### (6-Bromoisoquinolin-1-yl)methanol (14 d)

This compound was prepared from triazoloisoquinoline 13 d (298 mg, 1.2 mmol). The title compound was isolated as an off-white solid (238 mg, 83%). mp 143-145° C. ¹H NMR (400 MHZ, CDCl₃) δ: 8.47 (d, J=5.4 Hz, 1H), 8.03 (d, J=1.8 Hz, 1H), 7.79 (d, J=8.9 Hz, 1H), 7.69 (dd, J=8.9, 1.9 Hz, 1H), 7.51 (d, J=5.7 Hz, 1H), 5.20 (s, 2H), 4.89 (s, 1H). ¹³C NMR (101 MHZ, CDCl₃) δ: 158.2, 137.5, 131.6, 130, 125.7, 125.4, 123.8, 119.7, 61.8. HRMS (ESI-Q-TOF): m/z [M+H]⁺ calcd for C₁₀H₈BrNO: 237.9862; found: 237.9851

### Synthesis of 1-(chloromethyl)isoquinolines (15a-d)

### General Procedure

To a solution of 1-hydroxymethylisoquinoline 14a-d (1.2 mmol) in dry dichloromethane (1 ml) was added an excess of SOCl₂. The resulting reaction mixture was stirred for 3 hours at room temperature. After completion of the reaction, the reaction mixture was neutralized with an aqueous saturated NaHCO₃ solution. The aqueous phase was extracted several times with dichloromethane. The combined organic layers were dried over MgSO₄ and concentrated under vacuum to afford the title compound, that was used for the next step without further purification. The following compounds were made according to this procedure:

### 1-(Chloromethyl)-6,7-dimethoxyisoquinoline (15a)

This compound was prepared from 1-hydroxymethylisoquinoline 14a (263 mg, 1.2 mmol). The title compound was isolated as a red-orange solid; yield: 285 mg (99%); mp 148-150° C. ¹H NMR (400 MHZ, CDCl₃) δ: 8.34 (d, J=5.8 Hz, 1H), 7.50 (d, J=5.5 Hz, 1H), 7.42 (s, 1H), 7.09 (s, 1H), 5.09 (s, 2H), 4.07 (s, 3H), 4.03 (s, 3H). ¹³C NMR (101 MHZ, CDCl₃) δ: 153.1, 153.0, 150.6, 141.0, 133.8, 122.7, 120.7, 105.4, 103.2, 56.2, 56.2, 45.7. HRMS (ESI-Q-TOF): m/z [M+H]⁺ calcd for C₁₂H₁₂ClNO₂: 238.0629; found: 238.0631.

### 1-(Chloromethyl)-6-methoxyisoquinoline (15b)

This compound was prepared from 1-hydroxymethylisoquinoline 14b (227 mg, 1.2 mmol). The title compound was isolated as a dark red solid (225 mg, 90%). mp 85-87° C. ¹H NMR (400 MHZ, CDCl₃) δ: 8.40 (d, J=5.7 Hz, 1H), 8.15 (d, J=9.2 Hz, 1H), 7.55 (d, J=5.7 Hz, 1H), 7.29 (dd, J=9.2, 2.5 Hz, 1H), 7.10 (d, J=2.5 Hz, 1H), 5.09 (s, 2H), 3.96 (s, 3H). ¹³C NMR (101 MHZ, CDCl₃) δ: 160.9, 154.7, 141.7, 138.9, 126.8, 121.0, 120.8, 104.8, 55.4, 44.3. HRMS (ESI-Q-TOF): m/z [M+H]⁺ calcd for C₁₁H₁₀ClNO: 208.0524; found: 208.0516.

### 1-(Chloromethyl)-6-fluoroisoquinoline (15c)

This compound was prepared from 1-hydroxymethylisoquinoline 14c (213 mg, 1.2 mmol). The title compound was isolated as a red solid (224 mg, 95%). mp 65-67° C. ¹H NMR (600 MHZ, CDCl₃) δ: 8.47 (d, J=5.7 Hz, 1H), 8.30 (dd, J=9.1, 5.3 Hz, 1H), 7.62 (d, J=5.7 Hz, 1H), 7.52-7.39 (m, 2H), 5.12 (s, 2H). ¹³C NMR (151 MHZ, CDCl₃) δ: 164, 162.4, 155.9, 142.9, 138.6, 128.5, 123.8, 121.6, 118.5, 110.9, 45. HRMS (ESI-Q-TOF): m/z [M+H]⁺ calcd for C₁₀H₇ClFN: 196.0324; found: 196.0316.

### 6-Bromo-1-(chloromethyl)isoquinoline (15 d)

This compound was prepared from 1-hydroxymethylisoquinoline 14 d (286 mg, 1.2 mmol). The title compound was isolated as a redish solid (301 mg, 98%). mp 108-110° C. ¹H NMR (400 MHZ, CDCl₃) δ: 8.50 (d, J=5.7 Hz, 1H), 8.14 (d, J=9 Hz, 1H), 8.05 (d, J=1.9 Hz, 1H), 7.75 (dd, J=9, 1.9 Hz, 1H), 7.58 (d, J=7.6 Hz, 1H), 5.12 (s, 2H). ¹³C NMR (101 MHZ, CDCl₃) δ: 156.1, 143.1, 137.9, 131.5, 129.8, 127, 125.4, 125, 120.8, 44.8. HRMS (ESI-Q-TOF): m/z [M+H]⁺ calcd for C₁₀H₇BrClN: 255.9524; found: 255.9520

### tert-Butyl (4-aminobutyl) carbamate (17)

To a solution of 1,4-diaminobutane 16 (10 eq., 56.7 mmol, 5 g) in dichloromethane (62.5 mL) was added a solution of Boc₂O (1 eq., 5.67 mmol, 1.238 g) in dichloromethane (62.5 mL) through a dropping funnel. The reaction mixture was stirred at room temperature for 16 h. After filtering the suspension, the filtrate was evaporated under vacuum. The oily residue was washed with brine and extracted with EtOAc to remove the excess amount of diamine. The organic layer was dried over anhydrous Na₂SO₄ and evaporated under vacuum, yielding the title compound as a colourless oil (6.9 g, 65%). ¹H NMR (400 MHZ, CDCl₃) δ: 4.77 (s, 1H), 3.07 (t, J=6.0 Hz, 2H), 2.66 (t, J=6.6 Hz, 2H), 1.52-1.42 (m, 4H), 1.39 (s, 9H), 1.09 (s, 2H). ¹³C NMR (75 MHZ, CDCl₃) δ: 155.88, 78.41, 41.51, 40.09, 30.59, 28.17, 27.19. HRMS (ESI-Q-TOF): m/z [M+H]⁺ calcd for C₉H₂₀N₂O₂: 189.1597; found: 189.1597.

### tert-Butyl (4-((5,6,7,8-tetrahydroquinolin-8-yl)amino)butyl)carbamate (18)

6,7-Dihydroquinolin-8(5H)-one (1.03 eq., 3.40 mmol, 0.500 g) was added to a slurry of NaBH(OAc)₃ (1.78 eq., 5.87 mmol, 1.244 g) in dichloroethane (3 ml), followed by the addition of tert-butyl (4-aminobutyl) carbamate 17 (1 eq., 3.30 mmol, 0.621 g). The reaction was stirred at room temperature for 48 hours. The reaction mixture was quenched with a 1N NaOH solution to obtain a PH˜8 of the aqueous layer. The reaction mixture was extracted three times with dichloromethane (3×) and the combined organic layers were concentrated to a volume of approximately 3 ml. Heptane (10 ml) was added and the volume was concentrated to 5 ml. Upon cooling the reaction mixture to room temperature, a precipitate was formed. After further cooling the suspension to 0° C., the precipitate was filtered off and dried under vacuum, yielding the title compound as a light brown solid (674 mg, 64%); mp 73-75° C. ¹H NMR (400 MHZ, CDCl₃) δ: 8.39 (d, J=4.1 Hz, 1H), 7.82 (s, 1H), 7.41 (d, J=7.8 Hz, 1H), 7.12 (t, J=7.4, 4.9 Hz, 1H), 4.01 (s, 1H), 3.09 (t, J=33.0 Hz, 2H), 2.90-2.70 (m, 4H), 2.32-2.00 (m, 2H), 1.95 (t, J=16.3 Hz, 2H), 1.73-1.62 (m, 2H), 1.62-1.51 (m, 2H), 1.42 (s, 9H). ¹³C NMR (101 MHZ, CDCl₃) δ: 156.31, 147.03, 137.27, 132.95, 122.48, 57.79, 46.09, 28.64, 27.77, 27.40, 26.52, 22.02, 20.18. HRMS (ESI-Q-TOF): m/z [M+H]⁺ calcd for C₁₈H₂₉N₃O₂: 320.2332; found: 320.2335.

### Coupling of 1-(chloromethyl)isoquinolines 15a-d with amine 18

### General Procedure

To a solution of 1-(chloromethyl)isoquinoline 15a-d (1.5 eq) and tert-butyl (4-((5,6,7,8-tetrahydroquinolin-8-yl)amino)butyl)carbamate 18 (1 eq) in dry acetonitrile (5 ml) was added K₂CO₃ (8 eq). The reaction mixture was refluxed for 24-48 hours. The mixture was cooled to room temperature and filtered through a Celite® pad. The filtrate was evaporated in vacuo and the crude residue was purified via silica gel flash chromatography using a mixture of Et₂O/MeOH as mobile phase (in a gradient gradually ranging from 100:0 to 90:10) yielding the title compounds. The following compounds were made according to this procedure.

### tert-Butyl(4-(((6,7-dimethoxyisoquinolin-1-yl)methyl)(5,6,7,8-tetrahydroquinolin-8 yl) amino) butyl) carbamate

This compound was prepared from 1-(chloromethyl)-6,7-dimethoxyisoquinoline 15a (86 mg, 0.36 mmol) and tert-butyl (4-((5,6,7,8-tetrahydroquinolin-8-yl) amino) butyl) carbamate 18 (100 mg, 0.32 mmol). The title compound was isolated as a brown viscous oil (114 mg, 69%). ¹H NMR (300 MHZ, CDCl₃) δ: 8.52 (s, 1H), 8.43 (d, J=4.2 Hz, 1H), 8.26 (d, J=5.6 Hz, 1H), 7.37 (d, J=5.6 Hz, 1H), 7.30 (d, J=7.5 Hz, 1H), 7.03-6.97 (m, 2H), 4.62 (s, 1H), 4.35 (dd, J=56.5, 12.4 Hz, 2H), 4.14 (s, 3H), 4.00 (s, 3H), 2.97-2.46 (m, 6H), 2.12-1.90 (m, J=22.3, 11.4 Hz, 3H), 1.70-1.54 (m, 1H), 1.39 (s, 9H), 1.34-1.16 (m, J=28.5, 11.3 Hz, 4H). ¹³C NMR (75 MHZ, CDCl₃) δ: 156.09, 152.72, 149.61, 147.10, 140.57, 136.49, 134.38, 133.31, 124.39, 121.55, 119.29, 106.62, 104.75, 77.36, 60.90, 58.81, 56.51, 55.99, 50.53, 29.55, 28.54, 27.74, 25.31, 24.94, 21.80. HRMS (ESI-Q-TOF): m/z [M+H]+ calcd for C₃₀H₄₀N₄O₄: 521.3122; found: 521.3124.

### tert-Butyl(4-(((6-methoxyisoquinolin-1-yl)methyl)(5,6,7,8-tetrahydroquinolin-8-yl) amino) butyl) carbamate

This compound was prepared from 1-(chloromethyl)-6-methoxyisoquinoline 15b (104 mg, 0.33 mmol) and tert-butyl (4-((5,6,7,8-tetrahydroquinolin-8-yl)amino)butyl) carbamate 18 (101.4 mg, 0.49 mmol). The title compound was isolated as a brown viscous oil (136 mg, 85%). ¹H NMR (400 MHZ, CDCl₃) δ: 8.66 (d, J=9.2 Hz, 1H), 8.54 (d, J=3.9 Hz, 1H), 8.32 (d, J=5.8 Hz, 1H), 7.43 (d, J=5.8 Hz, 1H), 7.36 (d, J=7.4 Hz, 1H), 7.18 (dd, J=9.3, 2.5 Hz, 1H), 7.08 (dd, J=7.6, 4.6 Hz, 1H), 7.01 (d, J=2.6 Hz, 1H), 4.89 (s, 1H), 4.38 (s, 1H) 4.24 (s, 2H), 3.94 (s, 3H), 2.94-2.56 (m, 6H), 2.42-2.12 (m, 4H), 2.15-1.99 (m, 4H), 1.39 (s, 9H). ¹³C NMR (101 MHz, CDCl₃) δ: 161.5, 156.8, 147.8, 142.5, 139.1, 137.3, 135.2, 129.9, 122.5, 120.6, 120, 105, 56.1, 51.5, 40.5, 30.4, 30, 29.2, 28.2, 25.4, 22.2. HRMS (ESI-Q-TOF): m/z [M+H]⁺ calcd for C₂₉H₃₈N₄O₃: 491.3016; found: 491.3017.

### tert-Butyl(4-(((6-fluoroisoquinolin-1-yl)methyl)(5,6,7,8-tetrahydroquinolin-8-yl) amino) butyl) carbamate

This compound was prepared from 1-(chloromethyl)-6-fluoroisoquinoline 15c (117 mg, 0.6 mmol) and tert-butyl (4-((5,6,7,8-tetrahydroquinolin-8-yl)amino) butyl) carbamate 18 (127.8 mg, 0.4 mmol). The title compound was isolated as a brown viscous oil (164 mg, 86%). ¹H NMR (400 MHZ, CDCl₃) δ: 8.95 (dd, J=8.9, 5.8 Hz, 1H), 8.51 (d, J=3.9 Hz, 1H), 8.36 (d, J=5.7 Hz, 1H), 7.47 (d, J=5.7 Hz, 1H), 7.38-7.29 (m, 3H), 7.05 (dd, J=7.6, 4.7 Hz, 1H), 4.80 (s, 1H), 4.36 (d, J=12.5 Hz, 1H), 4.20 (d, J=3.6 Hz, 2H), 2.95-2.52 (m, 6H), 2.14-1.97 (m, 2H), 1.88-1.63 (m, 4H), 1.39 (s, 9H), 1.30-1.22 (m, 2H). ¹³C NMR (101 MHZ, CDCl₃) δ: 155.7, 146.8, 141.9, 137.7, 137.6, 136.3, 134.2, 130.8, 124.9, 121.4, 119.8, 109.6, 109.3, 78.5, 60.8, 57.2, 53.2, 50.6, 39.7, 33.3, 29.4, 29, 28.1, 27.2, 24.5, 24, 21.2. HRMS (ESI-Q-TOF): m/z [M+H]⁺ calcd for C₂₈H₃₅FN₄O₂: 479.2817; found: 479.2808.

### tert-Butyl (4-(((6-bromoisoquinolin-1-yl)methyl)(5,6,7,8-tetrahydroquinolin-8-yl) amino) butyl) carbamate

This compound was prepared from 1-(chloromethyl)-6-bromoisoquinoline 15 d (142 mg, 0.55 mmol) and tert-butyl (4-((5,6,7,8-tetrahydroquinolin-8-yl)amino) butyl) carbamate 18 (117.9 mg, 0.37 mmol). The title compound was isolated as a brown viscous oil (143 mg, 72%). ¹H NMR (400 MHZ, CDCl₃) δ: 8.78 (d, J=8.9 Hz, 1H), 8.53 (d, J=3.8 Hz, 1H), 8.40 (d, J=5.7 Hz, 1H), 7.92 (d, J=1.9 Hz, 1H), 7.64 (dd, J=9, 1.97 Hz, 1H), 7.43 (d, J=5.7 Hz, 1H), 7.35 (d, J=7.6 Hz, 1H), 7.06 (dd, J=7.6, 4.69 Hz, 1H), 4.80 (s, 1H), 4.36 (d, J=12, 1H), 4.20 (d, J=10 Hz, 2H), 2.95-2.54 (m, 6H), 2.11-2.03 (m, 2H), 1.75-1.58 (m, 4H), 1.41 (s, 9H), 1.21 (t, J=7 Hz, 2H). ¹³C NMR (101 MHZ, CDCl₃) δ: 155.9, 146.8, 141.9, 137.6, 137.3, 136.6, 130.2, 128.9, 128.7, 124.8, 123.2, 121.8, 119.3, 61.2, 57.1, 50.8, 39.6, 30.7, 29.3, 28.9, 28.2, 27.2, 27.5, 26.9, 24.3, 24, 21.2, 19.9. HRMS (ESI-Q-TOF): m/z [M+H]⁺ calcd for C₂₈H₃₅BrN₄O₂: 539.2016; found: 539.2021.

### tert-Butyl (4-((isoquinolin-1-ylmethyl)(5,6,7,8-tetrahydroquinolin-8-yl) amino) butyl)carbamate

This compound was prepared from the commercially available 1-(bromomethyl)isoquinoline (200 mg, 0.9 mmol) and tert-butyl (4-((5,6,7,8-tetrahydroquinolin-8-yl)amino)butyl) carbamate 18 (191.78 mg, 0.6 mmol). The title compound was isolated as a brown viscous oil (169 mg, 61%). ¹H NMR (400 MHZ, CDCl₃) δ: 8.52 (d, J=3.8 Hz, 1H), 8.09 (d, J=8.5 Hz, 1H), 8.00 (d, J=8.4 Hz, 1H), 7.94 (d, J=8.5 Hz, 1H), 7.77 (d, J=7.7 Hz, 1H), 7.68-7.61 (m, 1H), 7.52-7.43 (m, 1H), 7.33 (d, J=7.5 Hz, 1H), 7.04 (dd, J=7.6, 4.7 Hz, 1H), 4.86 (s, 1H), 4.21 (dd, J=8.3, 6.6 Hz, 1H), 4.01-3.87 (m, 2H), 2.99 (s, 2H), 2.86-2.64 (m, 4H), 2.11-1.87 (m, 2H), 1.54-1.44 (m, 2H), 1.42 (s, 9H), 1.29-1.20 (m, 2H). ¹³C NMR (101 MHz, CDCl₃) δ: 156.5, 147.7, 137.1, 136.6, 134.8, 129.6, 129.2, 128, 127.9, 126.3, 122.1, 121.9, 61.5, 58.9, 53.9, 52.8, 40.7, 30.2, 29.7, 28.9, 28.1, 26, 21.9. HRMS (ESI-Q-TOF): m/z [M+H]⁺ calcd for C₂₈H₃₆N₄O₂: 461.2911; found: 461.2935.

### 3-Methylpicolinaldehyde (22)

To a flame-dried flask under an inert argon atmosphere was added 2-bromo-3-methyl-pyridine 21 (1 eq., 11.40 mmol, 2 g) and Et₂O (1-2 ml) at −78° C., followed by addition of a 1.6M butyllithium solution in hexane (0.43 ml). After stirring for 3 hours at −78° C., DMF (2 ml) was added to this deep red-brown solution. The reaction temperature was gradually increased to room temperature and was stirred for an additional 22 hours. The reaction mixture was quenched through the dropwise addition of ice-water and extracted with Et₂O. The organic layer was washed with a 5% aqueous NaHCO₃ solution and brine. The combined organic layers were dried over MgSO₄ and concentrated under vacuum. The crude residue was purified by silica gel flash chromatography using Et₂O/petroleum ether (80:20) as the eluent, yielding the title compound as an orange-red oil (848 mg, 62%).

### tert-Butyl (4-(((3-methylpyridin-2-yl)methyl)amino)butyl) carbamate (23)

3-Methylpicolinaldehyde 22 (1 eq., 8.25 mmol, 1 g) was added to a slurry of NaBH(OAc)₃ (1.9 eq., 15.68 mmol, 3.324 g) in dichloroethane (15 ml), followed by the addition of tert-butyl (4-aminobutyl) carbamate 17 (1.4 eq., 11.56 mmol, 2.176 g). The reaction was stirred at room temperature for 48 hours. The reaction mixture was quenched with a 1N NaOH solution to obtain a pH˜8 in the aqueous layer. After extracting the mixture with dichloromethane (3×), the combined organic phases were concentrated to a volume of approximately 3 ml. The crude residue was purified by silica gel column chromatography using Et₂O/MeOH (96:4) as the eluent, affording the title compound as a yellowish viscous oil (1.8 g, 74%). ¹H NMR (400 MHZ, CDCl₃) δ: 8.37 (d, J=3.84 Hz, 1H), 7.41 (dd, J=7.6, 0.64 Hz, 1H), 7.06 (dd, J=7.5, 4.8 Hz, 1H), 4.79 (s, 1H), 3.86 (s, 2H), 3.17-3.06 (m, 2H), 2.94 (s, 1H), 2.72 (t, J=6.7 Hz, 2H), 2.29 (s, 3H), 1.64-1.49 (m, 4H), 1.42 (s, 9H). ¹³C NMR (101 MHz, CDCl₃) δ: 157.1, 156.1, 146.5, 137.7, 130.9, 121.9, 79, 52, 49.5, 40.5, 28.5, 27.4, 18.1. HRMS (ESI-Q-TOF): m/z [M+H]⁺ calcd for C₁₆H₂₇N₃O₂: 294.2176; found: 294.2169.

### Coupling of 1-(chloromethyl)isoquinolines 15a-d with amine 23

A similar procedure as mentioned earlier for the coupling with amine 18 was used. The following compounds were made according to this procedure.

### tert-Butyl(4-(((6,7-dimethoxyisoquinolin-1-yl)methyl)((3-methylpyridin-2 yl) methyl amino) butyl) carbamate

This compound was prepared from 1-(chloromethyl)-6,7-dimethoxyisoquinoline 15a (50 mg, 0.21 mmol) and tert-butyl (4-(((3-methylpyridin-2-yl) methyl)amino) butyl) carbamate 23 (48 mg, 0.15 mmol). The title compound was isolated as a brownish viscous oil (76 mg, 73%). ¹H NMR (400 MHZ, CDCl₃) δ: 8.38 (d, J=4.1 Hz, 1H), 8.30 (d, J=5.6 Hz, 1H), 7.51 (s, 1H), 7.42 (d, J=5.6 Hz, 1H), 7.36 (d, J=7.4 Hz, 1H), 7.08 (dd, J=7.6, 4.8 Hz, 1H), 7.02 (s, 1H), 4.75 (s, 1H), 4.23 (s, 2H), 4.00 (s, 3H), 3.82 (s, 5H), 2.96-2.83 (m, 2H), 2.59 (s, 2H), 2.10 (s, 3H), 1.64-1.51 (m, 2H), 1.40 (s, 9H), 1.30 (t, J=6.9 Hz, 2H). ¹³C NMR (101 MHZ, CDCl₃) δ: 162.5, 156.8, 155.9, 151.9, 151.3, 147.2, 141.2, 136.8, 133.1, 131.9, 123.9, 121.3, 117.7, 105.4, 79.5, 67.6, 59.1, 56.1, 54, 35.8, 28.4, 27.7, 25.8, 20.4. HRMS (ESI-Q-TOF): m/z [M+H]⁺ calcd for C₂₈H₃₈N₄O₄: 495.2966; found: 495.2960.

### tert-Butyl(4-(((6-methoxyisoquinolin-1-yl)methyl)((3-methylpyridin-2-yl) methyl) amino)butyl) carbamate

This compound was prepared from 1-(chloromethyl)-6-methoxyisoquinoline 15b (100 mg, 0.48 mmol) and tert-butyl (4-(((3-methylpyridin-2-yl)methyl)amino) butyl) carbamate 23 (94.2 mg, 0.32 mmol). The title compound was isolated as a brownish viscous oil (118 mg, 57%). ¹H NMR (400 MHZ, CDCl₃) δ: 8.38 (d, J=3.8 Hz, 1H), 8.33 (d, J=5.8 Hz, 1H), 7.88 (d, J=9 Hz, 1H), 7.43 (d, J=5.8 Hz, 1H), 7.40 (d, J=7.1 Hz, 1H), 7.10 (dd, J=7.5, 4.8 Hz, 1H), 7.06-6.98 (m, 2H), 4.71 (s, 1H), 4.14 (s, 2H), 3.91 (s, 3H), 3.83 (s, 2H), 2.94-2.82 (m, 2H), 2.57 (t, J=7.2 Hz, 2H), 2.10 (s, 3H), 1.58-1.46 (m, 2H), 1.44 (s, 9H), 1.32-1.17 (m, 2H). ¹³C NMR (101 MHZ, CDCl₃) δ: 160.2, 155.8, 146, 141.8, 138.2, 137.8, 133.2, 128.1, 123.2, 122.3, 119.7, 119.1, 104.1, 78.7, 59.1, 55.2, 53.7, 39.7, 28.2, 27.4, 22.7, 18. HRMS (ESI-Q-TOF): m/z [M+H]⁺ calcd for C₂₇H₃₆N₄O₃: 465.2860; found: 465.2863.

### tert-Butyl(4-(((6-fluoroisoquinolin-1-yl)methyl)((3-methylpyridin-2-yl) methyl) amino) butyl) carbamate

This compound was prepared from 1-(chloromethyl)-6-fluoroisoquinoline 15c (120 mg, 0.61 mmol) and tert-butyl (4-(((3-methylpyridin-2-yl) methyl)amino)butyl) carbamate 23 (120 mg, 0.41 mmol). The title compound was isolated as a brown viscous oil (150 mg, 81%). ¹H NMR (400 MHZ, CDCl₃) δ: 8.41 (d, J=5.7 Hz, 2H), 8.06 (dd, J=9.2, 5.7 Hz, 1H), 7.49 (d, J=5.8 Hz, 1H), 7.42 (d, J=6.9 Hz, 1H), 7.35 (dd, J=9.3, 2.5 Hz, 1H), 7.17 (dd, J=8.8, 2.4 Hz, 1H), 7.12 (dd, J=7.6, 4.9 Hz, 1H), 4.66 (s, 1H), 4.16 (s, 2H), 3.83 (s, 2H), 2.97-2.86 (m, 2H), 2.56 (t, J=7.3 Hz, 2H), 2.13 (s, 3H), 1.59-1.48 (m, 2H), 1.42 (s, 9H), 1.31-1.19 (m, 2H). ¹³C NMR (101 MHZ, CDCl₃) δ: 164.1, 161.6, 159, 156.9, 156.1, 146.4, 142.4, 138, 133.3, 129.9, 125, 122.6, 120.2, 116.9, 110, 79, 59.6, 59.4, 54.1, 40, 28.5, 27.8, 23, 18.3. HRMS (ESI-Q-TOF): m/z [M+H]⁺ calcd for C₂₆H₃₃FN₄O₂: 453.2660; found: 453.2651.

### tert-Butyl(4-(((6-bromoisoquinolin-1-yl)methyl)((3-methylpyridin-2-yl) methyl) amino) butyl) carbamate

This compound was prepared from 1-(chloromethyl)-6-bromoisoquinoline 15 d (100 mg, 0.39 mmol) and tert-butyl (4-(((3-methylpyridin-2-yl)methyl)amino)butyl) carbamate 23 (76.25 mg, 0.26 mmol). The title compound was isolated as a brown viscous oil (104 mg, 78%). ¹H NMR (400 MHZ, CDCl₃) δ: 8.43 (d, J=5.8 Hz, 1H), 8.40 (d, J=3.9 Hz, 1H), 7.93 (d, J=1.9 Hz, 1H), 7.85 (d, J=9 Hz, 1H), 7.50-7.41 (m, 3H), 7.12 (dd, J=7.6, 4.8 Hz, 1H), 4.65 (s, 1H), 4.15 (s, 2H), 3.83 (s, 2H), 2.96-2.87 (m, 2H), 2.56 (t, J=7.3 Hz, 2H), 2.16 (s, 3H), 1.75-1.60 (m, 2H), 1.58-1.47 (m, 2H), 1.42 (s, 9H). ¹³C NMR (101 MHZ, CDCl₃) δ: 157.1, 156.4, 146.7, 142.8, 138.4, 137.9, 133.7, 130.4, 129.3, 128.7, 126.5, 125, 123, 119.9, 79.3, 59.7, 54.5, 40.3, 28.8, 28.1, 23.3, 18.7. HRMS (ESI-Q-TOF): m/z [M+H]⁺ calcd for C₂₆H₃₃BrN₄O₂: 513.1860; found: 513.1854.

### tert-Butyl (4-((isoquinolin-1-ylmethyl)((3-methylpyridin-2-yl)methyl) amino) butyl) carbamate

This compound was prepared from 1-(bromomethyl)isoquinoline (100 mg, 0.45 mmol) and tert-butyl (4-(((3-methylpyridin-2-yl)methyl)amino) butyl)carbamate 15 (88 mg, 0.3 mmol). The title compound was isolated as a brown viscous oil (110 mg, 84%). ¹H NMR (400 MHZ, CDCl₃) δ: 8.37 (dd, J=4.7, 1.1 Hz, 1H), 8.05 (d, J=8.4 Hz, 2H), 7.77 (dd, J=8.2, 1.3 Hz, 1H), 7.71-7.65 (m, 1H), 7.54-7.47 (m, 2H), 7.39 (dd, J=7.7, 0.7 Hz, 1H), 7.06 (dd, J=7.6, 4.8 Hz, 1H), 4.74 (s, 1H), 3.91 (s, 2H), 3.86 (s, 2H), 3.06-2.93 (m, 2H), 2.57 (t, J=7.2 Hz, 2H), 2.33 (s, 3H), 1.70-1.61 (m, 2H), 1.59-1.50 (m, 2H), 1.42 (s, 9H). ¹³C NMR (101 MHZ, CDCl₃) δ: 161.2, 157.5, 156.4, 147.9, 146.6, 138.4, 136.4, 133.6, 129.7, 129.5, 127.9, 127.7, 126.5, 122.8, 121.9, 61.8, 60.1, 54.7, 40.6, 28.9, 28.2, 24.1, 19. HRMS (ESI-Q-TOF): m/z [M+H]⁺ calcd for C₂₆H₃₄N₄O₂: 435.2754; found: 435.2757.

### General Procedure for Deprotection of the Basic n-Butylamine Side Chain from Compounds 19a-e

### General Procedure

To a solution of the Boc-protected intermediates (0.1 mmol) in dry dichloromethane (1 ml) was added trifluoroacetic acid (0.3 ml). The reaction mixture was stirred for 1 hour at room temperature. The solvents were evaporated in vacuo. The residue was neutralized by adding a saturated aqueous NaHCO₃ solution and the aqueous layer was extracted five times with dichloromethane. The combined organic layers were dried over MgSO₄ and evaporated. The crude residue was purified via silica gel flash column chromatography using a mixture of DCM/MeOH (in a ratio gradually ranging from 95:5 to 90:10) as mobile phase. The following compounds were made according to this procedure.

### N1-((6,7-Dimethoxyisoquinolin-1-yl)methyl)-N1-(5,6,7,8-tetrahydroquinolin-8-yl) butane-1,4-diamine (19a)

This compound was prepared from its Boc-protected precursor (50 mg, 0.09 mmol). The title compound was isolated as a bright brown viscous oil (47 mg, 95%). ¹H NMR (400 MHZ, DMSO) δ (ppm): 8.43 (d, J=4.5 Hz, 1H), 8.21 (d, J=5.6 Hz, 1H), 8.17 (s, 1H), 7.55 (d, J=5.4 Hz, 1H), 7.48 (d, J=7.9 Hz, 1H), 7.30 (s, 1H), 7.20-7.10 (m, 1H), 5.32 (s, 1H), 4.29 (dd, J=21.5, 12.0 Hz, 2H), 3.99 (s, 3H), 3.92 (s, 3H), 2.15-1.50 (m, 6H), 1.39 (s, 2H), 1.30-1.09 (m, 4H), 1.07-0.93 (m, J=8.3, 6.1 Hz, 4H). ¹³C NMR (101 MHZ, DMSO) δ: 158.51, 158.20, 157.89, 157.59, 152.18, 139.94, 132.61, 123.25, 121.82, 121.65, 119.06, 118.84, 116.52, 115.85, 112.78, 55.51, 52.06, 49.46, 48.60, 45.67, 43.69, 7.17. HRMS (ESI-Q-TOF): m/z [M+H]+ calcd for C₂₅H₃₂N₄O₂: 421.2598; found: 421.2594.

### N1-((6-Methoxyisoquinolin-1-yl)methyl)-N1-(5,6,7,8-tetrahydroquinolin-8-yl)butane-1,4-diamine (19b)

This compound was prepared from its Boc-protected precursor (30 mg, 0.06 mmol). The title compound was isolated as a bright brown viscous oil (22 mg, 92%). ¹H NMR (400 MHZ, CDCl₃) δ: 9.99 (s, 2H), 8.54 (d, J=3.7 Hz, 1H), 8.51 (d, J=5.8 Hz, 1H), 8.03 (d, J=9.3 Hz, 1H), 7.51 (d, J=5.8 Hz, 1H), 7.41 (d, J=7.2 Hz, 1H), 7.22 (dd, J=9.3, 2.5 Hz, 1H), 7.12 (dd, J=7.7, 4.8 Hz, 1H), 7.08 (d, J=2.5 Hz, 1H), 4.47 (d, J=15 Hz, 1H), 4.05 (d, J=15 Hz, 2H), 3.07 (s, 3H), 3.01-2.46 (m, 6H), 2.12-1.91 (m, 4H), 1.81-1.54 (m, 4H). ¹³C NMR (101 MHZ, CDCl₃) δ: 161.1, 156.6, 156, 146.9, 141.6, 139, 138, 135, 125.9, 123, 122.7, 120.9, 120.4, 105.3, 60.2, 55.7, 51.6, 51.3, 39.7, 31.1, 29.3, 27.7, 26.9, 21.7, 21.12. HRMS (ESI-Q-TOF): m/z [M+H]+ calcd for C₂₄H₃₀N₄O: 391.2492; found: 391.2487.

### N1-((6-Fluoroisoquinolin-1-yl)methyl)-N1-(5,6,7,8-tetrahydroquinolin-8-yl)butane-1,4-diamine (19c)

This compound was prepared from its Boc-protected precursor (27 mg, 0.06 mmol). The title compound was isolated as a bright brown viscous oil (21 mg, 99%). ¹H NMR (400 MHZ, CDCl₃) δ: 9.84 (s, 2H), 8.60 (d, J=5.8 Hz, 1H), 8.52 (d, J=3.5 Hz, 1H), 8.19 (dd, J=9.3, 5.3 Hz, 1H), 7.57 (d, J=5.8 Hz, 1H), 7.47-7.32 (m, 3H), 7.11 (dd, J=7.6, 4.8 Hz, 1H), 4.46 (d, J=15.1 Hz, 1H), 4.11 (d, J=14.9 Hz, 2H), 2.98-2.52 (m, 6H), 2.12-1.94 (m, 4H), 1.82-1.58 (m, 4H). ¹³C NMR (101 MHZ, CDCl₃) δ: 164.5, 162, 157.3, 155.8, 147, 142.2, 138.2, 135, 127.4, 124.5, 122.8, 120.7, 118.3, 111.2, 60.3, 52.2, 51.5, 39.6, 29.8, 29.4, 27.7, 26.6, 21.7, 21.2. HRMS (ESI-Q-TOF): m/z [M+H]⁺ calcd for C₂₃H₂₇FN₄: 379.2292; found: 379.2286.

### N1-((6-Bromoisoquinolin-1-yl)methyl)-N1-(5,6,7,8-tetrahydroquinolin-8-yl)butane-1,4-diamine (19 d)

This compound was prepared from its Boc-protected precursor (22 mg, 0.04 mmol). The title compound was isolated as a bright brown viscous oil (18 mg, 99%). ¹H NMR (600 MHZ, CDCl₃) δ: 8.63 (d, J=5.8 Hz, 1H), 8.54 (d, J=3.7 Hz, 1H), 8.18 (d, J=8.9 Hz, 1H), 8.01 (d, J=1.8 Hz, 1H), 7.68 (dd, J=9, 1.9 Hz, 1H), 7.52 (d, J=5.8 Hz, 1H), 7.41 (d, J=7.2 Hz, 1H), 7.12 (dd, J=7.7, 4.7 Hz, 1H), 4.44 (d, J=14.8 Hz, 1H), 4.12 (d, J=14.9 Hz, 2H), 2.91-2.51 (m, 6H), 2.12-1.84 (m, 4H), 1.74-1.54 (m, 4H). ¹³C NMR (151 MHZ, CDCl₃) δ: 162.4, 158, 147.3, 14306, 136.8, 134.5, 132, 129.7, 128.4, 127.9, 125.5, 123.3, 121.3, 118.1, 67.7, 59.2, 54, 41.8, 28.5, 26.3, 25.8, 20.5. HRMS (ESI-Q-TOF): m/z [M+H]⁺ calcd for C₂₃H₂₇BrN₄: 439.1492; found: 439.1495.

### N1-(Isoquinolin-1-ylmethyl)-N1-(5,6,7,8-tetrahydroquinolin-8-yl)butane-1,4-diamine (26e)

This compound was prepared from its Boc-protected precursor (21 mg, 0.05 mmol). The title compound was isolated as a bright brown viscous oil (16 mg, 99%). ¹H NMR (400 MHZ, CDCl₃) δ: 10.04 (s, 2H), 8.61 (d, J=3.7 Hz, 1H), 8.20 (d, J=8.7 Hz, 1H), 8.15 (d, J=8.4 Hz, 1H), 7.79 (d, J=7.7 Hz, 2H), 7.58-7.51 (m, 1H), 7.40 (d, J=7.2 Hz, 1H), 7.33 (d, J=8.4 Hz, 1H), 7.13 (dd, J=7.6, 4.8 Hz, 1H), 4.06 (d, J=14.2 Hz, 2H), 3.92 (d, J=14.2 Hz, 1H), 3.03-2.67 (m, 6H), 2.13-1.89 (m, 4H), 1.76-1.53 (m, 4H). ¹³C NMR (101 MHZ, CDCl₃) δ: 162.4, 158.3, 147.3, 142.8, 136.8, 136.5, 132, 129.7, 127.5, 127.3, 126.8, 124.3, 121.3, 119.2, 67.7, 59.2, 54, 41.8, 28.5, 26.3, 25.8, 20.5. HRMS (ESI-Q-TOF): m/z [M+H]⁺ calcd for C₂₃H₂₈N₄: 361.2387; found: 361.2376.

### N1-((6,7-dimethoxyisoquinolin-1-yl)methyl)-N1-((3-methylpyridin-2-yl)methyl) butane-1,4-diamine (27a)

This compound was prepared from its Boc-protected precursor (17 mg, 0.03 mmol). The title compound was isolated as a bright brown viscous oil (13 mg, 99%). ¹H NMR (400 MHZ, CDCl₃) δ: 8.52 (d, J=4 Hz, 1H), 8.43 (d, J=5.6 Hz, 1H), 7.47 (d, J=5.6 Hz, 1H), 7.44 (d, J=6.9 Hz, 1H), 7.27 (s, 1H), 7.12 (dd, J=7.7, 4.9 Hz, 1H), 7.07 (s, 1H), 4.20 (s, 2H), 4.02 (s, 3H), 4.00 (s, 3H), 3.83 (s, 2H), 3.15 (t, J=5.4 Hz, 2H), 2.69 (t, J=5.3 Hz, 2H), 2.26 (s, 3H), 1.89-1.84 (m, 2H), 1.82-1.77 (m, 2H). ¹³C NMR (101 MHZ, CDCl₃) δ: 155.3, 153.9, 153, 150.4, 146.7, 140, 138.6, 1336, 131.8, 122.8, 122.6, 119.6, 105.4, 102.3, 57.1, 56.6, 561, 55.2, 39.5, 30.9, 29.6, 27.3, 26.2, 18.5. HRMS (ESI-Q-TOF): m/z [M+H]⁺ calcd for C₂₃H₃₀N₄O₂: 395.2441; found: 395.2434.

### N1-((6-Methoxyisoquinolin-1-yl)methyl)-N1-((3-methylpyridin-2-yl)methyl) butane-1,4-diamine (27b)

This compound was prepared from its Boc-protected precursor (22 mg, 0.05 mmol). The title compound was isolated as a bright brown viscous oil (17 mg, 99%). ¹H NMR (400 MHZ, CDCl₃) δ: 8.46 (dd, J=4.8, 1.1 Hz, 1H), 8.41 (d, J=5.8 Hz, 1H), 7.96 (d, J=9.3 Hz, 1H), 7.45 (d, J=5.8 Hz, 1H), 7.41 (dd, J=7.7, 0.7 Hz, 1H), 7.15 (dd, J=9.3, 2.6 Hz, 1H), 7.09 (dd, J=7.6, 4.8 Hz, 1H), 7.03 (d, J=2.5 Hz, 1H), 4.13 (s, 2H), 3.92 (s, 3H), 3.78 (s, 2H), 2.99-2.89 (m, 2H), 2.59 (t, J=5.7 Hz, 2H), 2.20 (s, 3H), 1.76-1.63 (m, 2H). ¹³C NMR (101 MHZ, CDCl₃) δ: 160.8, 156.9, 155.9, 146.7, 142, 138.7, 132.3, 126.6, 123, 122.7, 120.4, 120.2, 105, 57.7, 57.3, 55.6, 55.2, 39.8, 31.1, 28, 25.6, 18.6. HRMS (ESI-Q-TOF): m/z [M+H]⁺ calcd for C₂₂H₂₈N₄O: 365.2336; found: 365.2330.

### N1-((6-Fluoroisoquinolin-1-yl)methyl)-N1-((3-methylpyridin-2-yl)methyl) butane-1,4-diamine (27c)

This compound was prepared from its Boc-protected precursor (28 mg, 0.062 mmol). The title compound was isolated as a bright brown viscous oil (21.5 mg, 99%). 1H NMR (400 MHZ, CDCl₃) δ: 8.51 (d, J=5.8 Hz, 1H), 8.45 (d, J=3.8 Hz, 1H), 8.12 (dd, J=9.3, 5.4 Hz, 1H), 7.52 (d, J=5.8 Hz, 1H), 7.44-7.36 (m, 2H), 7.32 (td, J=8.9, 2.5 Hz, 1H), 7.08 (dd, J=7.6, 4.8 Hz, 1H), 4.22 (s, 2H), 3.82 (s, 2H), 3.01 (t, J=5.3 Hz, 2H), 2.69 (t, J=5.2 Hz, 2H), 2.22 (s, 3H), 1.86-1.69 (m, 4H). ¹³C NMR (101 MHZ, CDCl₃) δ: 163.9, 161.4, 158, 156, 146.3, 142.2, 137.9, 132.5, 128.7, 124.5, 122.4, 120.1, 117.1, 110.2, 58.3, 54.7, 40.5, 30.7, 29.5, 24.2, 18.2. HRMS (ESI-Q-TOF): m/z [M+H]⁺ calcd for C₂₁H₂₅FN₄: 353.2136; found: 353.2131.

### N1-((6-Bromoisoquinolin-1-yl)methyl)-N1-((3-methylpyridin-2-yl)methyl) butane-1,4-diamine (27 d)

This compound was prepared from its Boc-protected precursor (22 mg, 0.043 mmol). The title compound was isolated as a bright brown viscous oil (17.6 mg, 99%). 1H NMR (400 MHZ, CDCl₃) δ: 8.57 (d, J=5.7 Hz, 1H), 8.46 (d, J=3.9 Hz, 1H), 7.97 (d, J=1.8 Hz, 1H), 7.94 (d, J=9.1 Hz, 1H), 7.63 (dd, J=9, 1.9 Hz, 1H), 7.47 (d, J=5.8 Hz, 1H), 7.41 (d, J=7.7 Hz, 1H), 7.09 (dd, J=7.6, 4.8 Hz, 1H), 4.19 (s, 2H), 3.82 (s, 2H), 3.04 (t, J=4.7 Hz, 2H), 2.68 (t, J=5.5 Hz, 2H), 2.22 (s, 3H), 1.87-1.70 (m, 4H). ¹³C NMR (101 MHZ, CDCl₃) δ: 157.3, 155.2, 146.5, 142.3, 138.4, 137.3, 131.9, 130.8, 129.5, 126.1, 125.4, 125, 122.4, 119.5, 56.9, 55, 39.3, 30.8, 29.5, 27.1, 25.3, 18.4. HRMS (ESI-Q-TOF): m/z [M+H]⁺ calcd for C₂₁H₂₅BrN₄: 413.1336; found: 413.1339.

### N1-(Isoquinolin-1-ylmethyl)-N1-((3-methylpyridin-2-yl)methyl)butane-1,4-diamine (27e)

This compound was prepared from its Boc-protected precursor (22 mg, 0.05 mmol). The title compound was isolated as a bright brown viscous oil (17 mg, 99%). ¹H NMR (400 MHZ, CDCl₃) δ: 8.43 (dd, J=4.8, 1.1 Hz, 1H), 8.08 (dd, J=8.3, 4.7 Hz, 2H), 7.77 (dd, J=8.1, 1.1 Hz, 1H), 7.74-7.68 (m, 1H), 7.54-7.49 (m, 1H), 7.47 (d, J=8.5 Hz, 1H), 7.41 (dd, J=7.6, 0.9 Hz, 1H), 7.08 (dd, J=7.6, 4.8 Hz, 1H), 3.92 (s, 2H), 3.85 (s, 2H), 2.75 (t, J=6.5 Hz, 2H), 2.58 (t, J=6.8 Hz, 2H), 2.35 (s, 3H), 1.65-1.56 (m, 2H), 1.53-1.43 (m, 2H). ¹³C NMR (101 MHZ, CDCl₃) δ: 158.1, 155.1, 147.2, 146.4, 138.4, 137.1, 131.7, 130.2, 128.2, 127.4, 127.1, 126.5, 122.4, 122.3, 61, 56.9, 55, 39.2, 30.7, 29.5, 26.9, 25.6, 18.3. HRMS (ESI-Q-TOF): m/z [M+H]+ calcd for C₂₁H₂₆N₄: 335.2230; found: 335.2228.

### tert-Butyl (4-((pyridin-2-ylmethyl)(5,6,7,8-tetrahydroquinolin-8-yl)amino) butyl)carbamate

Picolinaldehyde (1 eq., 0.6887 mmol, 0.0655 ml) was added to a slurry of NaBH(OAc)₃ (1.78 eq., 1.2259 mmol, 259.8 mg) in dichloromethane (3 ml), followed by the addition of tert-butyl (4-((5,6,7,8-tetrahydroquinolin-8-yl)amino) butyl)carbamate 18 (1 eq., 0.6887 mmol, 220 mg). The reaction mixture was stirred at room temperature for 48 hours. The, the reaction was quenched with a 1N NaOH solution to obtain pH˜8 of the aqueous layer. The aqueous layer was extracted three times with dichloromethane. The combined organic phases were concentrated and the crude residue was purified via silica gel flash chromatography using a mixture of DCM/MeOH (in a ratio of 96:4) as mobile phase, affording the title compound as a brown viscous oil (177 mg, 63%). ¹H NMR (400 MHZ, CDCl₃) δ: 8.49 (d, J=3.7 Hz, 1H), 8.45 (d, J=4.8 Hz, 1H), 7.73 (d, J=7.9 Hz, 1H), 7.63, (td, J=7.5, 1.7 Hz, 1H), 7.33 (d, J=7.5 Hz, 1H), 7.10 (dd, J=6.5, 5.6 Hz, 1H), 7.04 (dd, J=7.6, 4.7 Hz, 1H), 4.8 (s, 1H), 4.13 (d, J=7.4 Hz, 1H), 3.91 (d, J=14.6 Hz, 2H), 3.09-2.94 (m, 2H), 2.86-2.63 (m, 4H), 2.22-2.09 (m, 2H), 2.06-1.95 (m, 2H), 1.94-1.81 (m, 2H), 1.76-1.61 (m, 2H), 1.41 (s, 9H). ¹³C NMR (101 MHZ, CDCl₃) δ: 162.4, 157.9, 155.9, 148.7, 147.3, 139.7, 136.7, 132, 124.2, 121.2, 121, 79.5, 67.7, 60.9, 54, 35.9, 28.4, 27.7, 25.8, 20.5. HRMS (ESI-Q-TOF): m/z [M+H]⁺ calcd for C₂₄H₃₄N₄O₂: 411.2754; found: 411.2757.

### tert-Butyl (4-(((3-methylpyridin-2-yl)methyl)(pyridin-2-ylmethyl) amino) butyl) carbamate

Picolinaldehyde (1 eq., 1.02 mmol, 0.1 ml) was added to a slurry of NaBH(OAc)₃ (1·78 eq., 1.82 mmol, 385.7 mg) in DCM (3 ml), followed by addition of tert-butyl (4-(((3-methylpyridin-2-yl)methyl)amino)butyl)carbamate 23 (1 eq., 1.02 mmol, 200 mg). The reaction was stirred at room temperature for 48 hours. After completion, the reaction mixture was quenched utilizing 1N NaOH solution to obtain pH˜8 in the aqueous layer. After extracting the mixture with DCM for 3 times, the combined organic phases were concentrated. Thereafter, the residue was purified through column chromatography using a DCM/MeOH (96:4) gradient elution to afford the corresponding compound. Brown viscous oil; yield: 206 mg (52%). ¹H NMR (400 MHz, CDCl₃) δ: 8.50 (d, J=4.1 Hz, 1H), 8.36 (dd, J=4.8, 1.2 Hz, 1H), 7.59 (td, J=7.7, 1.8 Hz, 1H), 7.39 (d, J=6.7 Hz, 1H), 7.34 (d, J=10.3 Hz, 1H), 7.12 (dddd, J=7.4, 5, 1.1 Hz, 1H), 7.07 (dd, J=7.6, 4.8 Hz, 1H), 4.81 (s, 1H), 3.80 (s, 2H), 3.75 (s, 2H), 3.06-2.92 (m, 2H), 2.53 (t, J=7.2 Hz, 2H), 2.30 (s, 3H), 1.57-1.47 (m, 2H), 1.43 (s, 9H), 1.41-1.31 (m, 2H). ¹³C NMR (101 MHZ, CDCl₃) δ: 160.1, 157.3, 156.4, 149.1, 146.4, 138.3, 136.5, 133.5, 123.7, 122.7, 122.1, 60.7, 59.9, 54.4, 40.4, 28.8, 28.1, 24, 18.7. HRMS (ESI-Q-TOF): m/z [M+H]⁺ calcd for C₂₂H₃₂N₄O₂: 385.2598; found: 385.2591.

### N1-(Pyridin-2-ylmethyl)-N1-(5,6,7,8-tetrahydroquinolin-8-yl)butane-1,4-diamine (20)

This compound was synthesized starting from its Boc-protected precursor (23 mg, 0.06 mmol), according to the general deprotection procedure, yielding the title compound as a brownish viscous oil (17 mg, 99%). ¹H NMR (600 MHZ, CDCl₃) δ: 9.91 (s, 2H), 8.73 (dd, J=4.9, 0.86 Hz, 1H), 8.58 (d, J=3.9 Hz, 1H), 7.68 (td, J=7.7, 1.8 Hz, 1H), 7.41 (d, J=7 Hz, 1H), 7.29-7.21 (m, 2H), 7.15 (dd, J=7.7, 4.8 Hz, 1H), 3.92 (d, J=13.7 Hz, 2H), 3.73 (d, J=13.7 Hz, 1H), 2.99-2.64 (m, 4H), 2.39-2.20 (m, 2H), 2.11-1.92 (m, 2H), 1.75-1.56 (m, 6H). ¹³C NMR (151 MHZ, CDCl₃) δ: 162.5, 157.9, 148.7, 17.2, 139.8, 136.7, 131.9, 124.2, 121.3, 121, 67.7, 60.9, 53.9, 41.8, 28.4, 26.3, 25.8, 20.5. HRMS (ESI-Q-TOF): m/z [M+H]⁺ calcd for C₁₉H₂₆N₄: 311.2230; found: 311.2226.

### N1-((3-Methylpyridin-2-yl)methyl)-N1-(pyridin-2-ylmethyl)butane-1,4-diamine (25)

This compound was synthesized starting from its Boc-protected precursor (30 mg, 0.08 mmol), according to the general deprotection procedure, yielding the title compound as a brownish viscous oil (18 mg, 81%). ¹H NMR (600 MHZ, CDCl₃) δ: 9.36 (s, 2H), 8.66 (d, J=4.5 Hz, 1H), 8.52 (d, J=4.5 Hz, 1H), 7.65 (td, J=7.7, 1.6 Hz, 1H), 7.43 (d, J=7.5 Hz, 1H), 7.21 (d, J=7.4 Hz, 1H), 7.20 (d, J=5.1 Hz, 1H), 7.12 (dd, J=7.5, 4.9 Hz, 1H), 3.77 (s, 2H), 3.73 (s, 2H), 3.13 (t, J=5.4 Hz, 2H), 2.58 (t, J=5.2 Hz, 2H), 2.25 (s, 3H), 1.85-1.79 (m, 2H), 1.78-1.73 (m, 2H). ¹³C NMR (151 MHZ, CDCl₃) δ: 157.4, 1552, 149.8, 146.8, 138.9, 137.4, 132, 124, 123.1, 122.8, 60.4, 57, 55.1, 39.6, 29.8, 27.3, 262, 18.6. HRMS (ESI-Q-TOF): m/z [M+H]⁺ calcd for C₁₇H₂₄N₄: 285.2074; found: 285.2077.

### 2-(4-Hydroxybutyl)isoindoline-1,3-dione (27)

To a reaction flask equipped with a stirring bar and a Dean-Stark receiver, containing 4-aminobutan-1-ol 26 (1.78 g, 20.0 mmol, 1.0 eq.) in toluene (60 ml) phthalic anhydride (2.96 g, 20.0 mmol, 1.0 eq.) was added. The mixture was refluxed for 3.5 h. The solvent was further evaporated in vacuo to give the title compound (4.25 g, 19.0 mmol, 97%) as a white solid. ¹H NMR (400 MHZ, CDCl₃) δ (ppm): 7.87-7.82 (m, 2H), 7.74-7.69 (m, 2H), 3.74 (t, J=7.1 Hz, 2H), 3.69 (t, J=6.4, 2H), 1.82-1.75 (m, 2H), 1.69 (s br, 1H), 1.65-1.59 (m, 2H).

### 4-(1,3-Dioxoisoindolin-2-yl) butanal (28)

In a flask equipped with a stirring bar, oxalyl chloride (0.34 mL, 4.0 mmol, 2.0 eq.) was dissolved in dichloromethane (15 mL) and cooled down to −78° C. To the mixture, a solution of DMSO (0.57 mL, 8.0 mmol, 4.0 eq.) in dichloromethane (4 mL) was added over 30 min. Upon addition, the mixture was stirred at −78° C. for 5 min, followed by the addition of a solution of the alcohol 27 (0.44 g, 2.0 mmol, 1.0 eq.) in dichloromethane (4 mL) over 30 min at −78° C. The resulting mixture was stirred for 40 min, after which Et₃N (1.7 mL, 12.0 mmol, 6.0 eq.) was added dropwise over 10 min. The resulting mixture was allowed to warm up to 0° C. and stirred at 0° C. for 1 h. The reaction was quenched with water (10 mL). The organic layer was then separated and further washed twice with water (15 mL) and brine. The combined organic layers were dried over anhydrous magnesium sulfate and concentrated, yielding a pale yellow solid (2.370 g, 10.9 mmol, 99%). ¹H NMR (400 MHZ, CDCl₃) δ (ppm): 9.77 (t, J=1.1 Hz, 1H), 7.87-7.83 (m, 2H), 7.75-7.70 (m, 2H), 3.75 (t, J=6.8 Hz, 2H), 2.54 (dt, J=1.1, 7.3 Hz, 2H), 2.02 (quint, J=6.8 Hz, 2H).

### 2-(4-(Quinolin-8-ylamino)butyl)isoindoline-1,3-dione (29)

To an oven-dried, Ar-flushed, screw-capped reaction tube equipped with a magnetic stirring bar, 4-(1,3-dioxoisoindolin-2-yl)butanal 27 (1.63 g, 7.5 mmol, 1.5 eq.), 8-quinolinamine (0.721 g, 5.000 mmol, 1.000 eq.) and sodium triacetoxyborohydride (2.86 g, 13.5 mmol, 2.7 eq.) were added. The reaction tube was evacuated and backfilled with argon three times. The mixture was dissolve in dry dichlorethane (17 mL) after which acetic acid (1.7 mL, 30.0 mmol, 6.0 eq.) was gradually added. The resulting mixture was stirred at room temperature under inert atmosphere for 20.5 h. The reaction was carefully quenched with aqueous NaHCO₃ (15 mL) and extracted three times with dichloromethane (30 mL). The combined organic layers were dried over anhydrous magnesium sulfate, filtered and concentrated in vacuo. Silica gel column chromatography of the crude product using isohexanes/ethyl acetate (4/1) as the mobile phase provided the title compound as a yellow solid (1.27 g, 3.7 mmol, 74%). MP 93.8-95.0° C. ¹H NMR (400 MHZ, CDCl₃) δ (ppm): 8.69 (dd, J=1.7, 4.3 Hz, 1H), 8.03 (dd, J=1.7, 8.4 Hz, 1H), 7.86-7.81 (m, 2H), 7.72-7.67 (m, 2H), 7.37-7.33 (m, 2H), 7.02 (dd, J=0.8, 8.3 Hz, 1H), 6.65 (d, J=7.6 Hz, 1H), 6.12 (s br, 1H), 3.79-3.75 (m, 2H), 3.38-3.34 (m, 2H), 1.93-1.79 (m, 4H). ¹³C NMR (75 MHZ, CDCl₃) δ (ppm): 168.4, 146.7, 144.7, 138.1, 135.9, 132.1, 128.6, 127.7, 121.3, 113.7, 104.5, 42.9, 37.7, 26.6, 26.4. HRMS (ESI-Q-TOF):m/z[M+H]⁺ calcd for C₂₁H₁₉N₃O₂: 346.15499; found: 346.1551

### General Procedure for the Synthesis of Protected N1-substituted-N1-(quinolin-8-yl)butane-1,4-diamines (30a-d)

To an oven-dried, Ar-flushed, screw-capped reaction tube equipped with a magnetic stirring bar, 2-(4-(quinolin-8-ylamino)butyl)isoindoline-1,3-dione 28 (1.0 eq.), the benzyl halide derivative (1.5 eq.), potassium carbonate (8.0 eq.) and sodium iodide (0.250 eq.) were added. The reaction tube was evacuated and backfilled with argon three times. The mixture was dissolved in dry acetonitrile (67 mM) and refluxed up to 24 h. The mixture was cooled to room temperature and filtered through a Celite pad. The filtrate was evaporated in vacuo, basic alumina column chromatography of the crude product using isohexanes/ethyl acetate (varying between 4/1 and 3/2) as the mobile phase provided the corresponding protected/1-substituted-N1-(quinolin-8-yl)butane-1,4-diamines as semi-solids.

### 2-(4-(Benzyl(quinolin-8-yl)amino)butyl)isoindoline-1,3-dione (30a)

This compound was prepared according to general procedure using benzyl bromide (0.036 mL, 0.300 mmol), intermediate 29 (0.069 g, 0.20 mmol), potassium carbonate (0.221 g, 1.60 mmol) and dry acetonitrile (3 mL). Silica gel column chromatography of the crude product using isohexanes/ethyl acetate (3/2) as the mobile phase provided the title compound as a yellow viscous oil (0.062 g, 0.14 mmol, 71%). ¹H NMR (400 MHZ, CDCl₃) δ(ppm): 8.89 (dd, J=1.2, 3.9 Hz, 1H), 8.09 (dd, J=1.6, 8.2, 1H), 7.81-7.77 (m, 2H), 7.71-7.66 (m, 2H), 7.38-7.30 (m, 3H), 7.23-7.15 (m, 5H), 7.01-6.96 (m, 1H), 4.74 (s, 1H), 3.62-3.59 (m, 2H), 3.48-3.41 (m, 2H), 1.69-1.63 (m, 4H). ¹³C NMR (75 MHZ, CDCl₃) δ(ppm): 168.3, 147.7, 147.1, 143.2, 138.7, 136.4, 133.8, 132.1, 129.8, 128.5, 128.0, 126.7, 126.3, 123.1, 120.8, 120.6, 118.6, 58.0, 51.7, 37.9, 26.3, 24.2. HRMS (ESI-Q-TOF): m/z[M+H]⁺ calcd for C₂₈H₂₅N₃O₂: 436.20194; found: 436.2003.

### 2-(4-((Pyridin-2-ylmethyl)(quinolin-8-yl)amino)butyl)isoindoline-1,3-dione (30b)

This compound was prepared according to the general procedure using 2-(chloromethyl)pyridine hydrochloride (0.049 g, 0.30 mmol), intermediate 29 (0.069 g, 0.20 mmol), potassium carbonate (0.221 g, 1.6 mmol), sodium iodide (0.007 g, 0.05 mmol) and dry acetonitrile (3 mL). Silica gel column chromatography of the crude product using isohexanes/ethyl acetate (4/1) as the mobile phase provided the title compound as a yellow viscous oil (0.044 g, 0.10 mmol, 50%). ¹H NMR (400 MHz, CDCl₃) ð (ppm): 8.86 (dd, J=1.6, 4.1 Hz, 1H), 8.51-8.50 (m, 1H), 8.08 (dd, J=1.6, 8.3 Hz, 1H), 7.81-7.77 (m, 2H), 7.71-7.66 (m, 2H), 7.51 (dt, J=1.4, 7.5 Hz, 1H), 7.46-7.41 (m, 1H), 7.38-7.30 (m, 3H), 7.11-7.03 (m, 2H), 4.84 (s, 2H), 3.64-3.57 (m, 4H), 1.74-1.61 (m, 4H). ¹³C NMR (75 MHZ, CDCl₃) 0 (ppm): 168.3, 159.7, 148.7, 147.7, 146.8, 143.1, 136.4, 136.3, 133.7, 132.1, 129.8, 126.4, 123.1, 122.7, 121.8, 120.8, 120.6, 118.1, 59.6, 53.1, 37.9, 26.3, 24.3. HRMS (ESI-Q-TOF):m/z[M+H]⁺ calcd for C₂₇H₂₄N₄O₂: 437.19719; found: 437.1966

### 2-(4-((Isoquinolin-1-ylmethyl)(quinolin-8-yl)amino)butyl)isoindoline-1,3-dione (30c)

This compound was prepared according to general procedure using 1-(chloromethyl)isoquinoline hydrochloride (0.096 g, 0.45 mmol, intermediate 29 (0.104 g, 0.30 mmol), potassium carbonate (0.332 g, 2.4 mmol), sodium iodide (0.011 g, 0.075 mmol), dry acetonitrile (4.5 mL). Silica gel column chromatography of the crude product using isohexanes/ethyl acetate (3/2) as the mobile phase provided the title compound as a yellow viscous oil (0.093 g, 0.19 mmol, 64%). ¹H NMR (400 MHZ, CDCl₃) & (ppm): 8.88 (dd, J=2.3, 5.9 Hz, 1H), 8.54 (d, J=8.6 Hz, 1H), 8.42 (d, J=5.7 Hz, 1H), 8.09 (dd, J=1.8, 8.3 Hz, 1H), 7.78-7.76 (m, 2H), 7.73 (d, J=8.5 Hz, 1H), 7.68-7.65 (m, 2H), 7.57-7.53 (m, 1H), 7.51 (d, J=5.7 Hz, 1H), 7.39-7.33 (m, 4H), 7.21 (dd, J=2.6, 6.5 Hz, 1H), 5.30 (s, 2H), 3.58 (m, 4H), 1.64-1.52 (m, 4H)

### 2-(4-(((6-Fluoroisoquinolin-1-yl)methyl) (quinolin-8-yl)amino)butyl)isoindoline-1,3-dione (30 d)

This compound was prepared according to the general procedure using 1-(chloromethyl)-6-fluoroisoquinoline hydrochloride (0.139 g, 0.60 mmol), intermediate 29 (0.138 g, 0.40 mmol), potassium carbonate (0.442 g, 3.200 mmol), sodium iodide (0.015 g, 0.10 mmol), dry acetonitrile (6.0 mL). Silica gel column chromatography of the crude product using isohexanes/ethyl acetate (3/2) as the mobile phase provided the title compound as a yellow viscous oil (0.109 g, 0.22 mmol, 54%).

¹H NMR (400 MHZ, CDCl₃) δ (ppm): 8.88 (dd, J=1.8, 4.1 Hz, 1H), 8.85 (dd, J=5.7, 9.3 Hz, 1H), 8.41 (d, J=5.7 Hz, 1H), 8.10 (dd, J=1.8, 8.4 Hz, 1H), 7.77-7.74 (m, 2H), 7.69-7.65 (m, 2H), 7.48 (d, J=5.7 Hz, 1H), 7.39-7.31 (m, 4H), 7.22 (dd, J=2.0, 6.9 Hz, 1H), 7.13 (td, J=2.0, 13.5 Hz, 1H), 5.24 (s, 2H), 3.53-3.47 (m, 4H), 1.64-1.48 (m, 4H). ¹³C NMR (75 MHz, CDCl₃) δ (ppm): 168.3, 162.8 (d, J=252.1 Hz), 158.6, 147.9, 147.3, 143.6, 142.4, 138.1 (d, J=10.3 Hz), 136.5, 133.8, 132.1, 130.4 (d, J=9.4 Hz), 129.8, 126.5, 125.2, 123.1, 121.5, 120.9, 120.1, 120.0, 117.0 (d, J=24.7 Hz), 109.8 (d, J=20.5 Hz), 59.5, 51.8, 37.8, 26.3, 23.7. HRMS (ESI-Q-TOF):m/z [M+H]⁺ calcd for C₃₁H₂₅FN₄O₂: 505.20341; found: 505.2029.

### General Procedure for the Synthesis of N¹-substituted-N¹-(quinolin-8-yl)butane-1,4-diamines (31a-d)

To a reaction tube equipped with a stirring bar, containing protected/1-substituted-N¹-(quinolin-8-yl)butane-1,4-diamine (1.0 eq.) in ethanol (1.5 mL), hydrazine monohydrate (10 eq.) was added. The mixture was refluxed for 2 h. Upon completion and cooling, EtOH was evaporated in vacuo. Water (5 mL) was added to the remaining residue and further extracted 3 times with dichloromethane (10 mL). The combined organic layers were dried over anhydrous magnesium sulfate and concentrated to afford the corresponding N¹-substituted-N¹-(quinolin-8-yl)butane-1,4-diamines as semi-solids. No further purification was performed unless stated otherwise.

N¹-benzyl-N¹-(quinolin-8-yl)butane-1,4-diamine (31a)

This compound was prepared according to the general procedure using 2-(4-(Benzyl(quinolin-8-yl)amino)butyl)isoindoline-1,3-dione 30a (0.044 g, 0.10 mmol), hydrazine monohydrate (50 μL), ethanol (1.5 mL) affording the desired compound as a bright yellow semi-solid (0.030 g, 0.10 mmol, 98%). ¹H NMR (400 MHZ, CDCl₃) δ (ppm): 8.91 (dd, J=1.8, 4.1 Hz, 1H), 8.11 (dd, J=1.8, 8.2 Hz, 1H), 7.39-7.32 (m, 3H), 7.24-7.17 (m, 5H), 6.99 (dd, J=2.4, 6.7, 1H), 4.76 (s, 2H), 3.43-3.39 (m, 2H), 2.63-2.60 (m, 2H), 1.69-1.61 (m, 2H), 1.44-1.37 (m, 2H), 1.25 (s br, 1H). ¹³C NMR (75 MHZ, CDCl₃) δ (ppm): 147.9, 147.7, 147.1, 143.3, 138.6, 136.5, 129.9, 128.7, 128.0, 126.8, 126.3, 120.9, 120.7, 118.8, 58.2, 51.9, 41.9, 31.2, 24.2. HRMS (ESI-Q-TOF):m/z[M+H]⁺ calcd for C₂₀H₂₃N₃: 306.19646; found: 306.1961.

### N¹-(pyridin-2-ylmethyl)-N¹-(quinolin-8-yl)butane-1,4-diamine (31b)

This compound was prepared according to the general procedure using 2-(4-((pyridin-2-ylmethyl)(quinolin-8-yl)amino)butyl)isoindoline-1,3-dione 30b (0.044 g, 0.10 mmol), hydrazine monohydrate (50 μL), ethanol (1.5 mL) affording the desired compound as a bright yellow semi-solid (0.030 g, 0.10 mmol, 97%). ¹H NMR (400 MHz, CDCl₃) δ(ppm): 8.89 (dd, J=1.8, 4.2 Hz, 1H), 8.55-8.52 (m, 1H), 8.11 (dd, J=1.8, 8.2 Hz, 1H), 7.52 (dt, J=1.8, 7.6 Hz, 1H), 7.42-7.34 (m, 4H), 7.13-7.06 (m, 2H), 4.84 (s, 2H), 3.57-3.51 (m, 2H), 2.67 (t, J=6.9 Hz, 2H), 1.74-1.66 (m, 2H), 1.50-1.41 (m, 2H), 1.25 (s, 2H). ¹³C NMR (75 MHZ, CDCl₃) δ (ppm): 159.5, 148.8, 147.8, 146.9, 143.1, 136.5, 136.3, 129.9, 126.4, 122.8, 121.8, 120.9, 120.8, 118.2, 59.5, 53.4, 41.8, 29.7, 24.3. HRMS (ESI-Q-TOF):m/z[M+H]⁺ calcd for C₁₉H₂₂N₄: 307.19171; found: 307.1912.

### N¹-(isoquinolin-1-ylmethyl)-N¹-(quinolin-8-yl)butane-1,4-diamine (31c)

This compound was prepared according to the general procedure using 2-(4-((isoquinolin-1-ylmethyl) (quinolin-8-yl)amino) butyl)isoindoline-1,3-dione 30c (0.053 g, 0.11 mmol), hydrazine monohydrate (53 μL), ethanol (1.6 mL) affording the desired compound as a bright yellow semi-solid (0.037 g, 0.10 mmol, 96%). 1H NMR (400 MHZ, CDCl₃) δ (ppm): 8.91 (dd, J=1.8, 4.1 Hz, 1H), 8.53 (d, J=8.5 Hz, 1H), 8.47 (d, J=5.8 Hz, 1H), 8.12 (dd, J=1.8, 8.3 Hz, 1H), 7.77 (d, J=8.3 Hz, 1H), 7.61-7.56 (m, 1H), 7.55 (d, J=5.8 Hz, 1H), 7.41-7.35 (m, 4H), 7.22 (dd, J=1.9, 6.9 Hz, 1H), 5.31 (s, 2H), 3.52 (t, J=7.6 Hz, 1H), 2.53 (t, J=6.9 Hz, 1H), 1.67-1.59 (m, 2H), 1.36-1.28 (m, 2H). ¹³C NMR (75 MHZ, CDCl₃) δ (ppm): 158.1, 148.0, 147.6, 143.5, 141.4, 136.7, 136.4, 129.9, 127.7, 126.9, 126.8, 126.5, 126.2, 121.5, 120.9, 120.5, 119.6, 58.2, 52.4, 41.5, 30.7, 23.9.

### N¹-((6-fluoroisoquinolin-1-yl)methyl)-N¹-(quinolin-8-yl)butane-1,4-diamine (31 d)

This compound was prepared according to the general procedure using 2-(4-(((6-fluoroisoquinolin-1-yl) methyl)(quinolin-8-yl)amino) butyl)isoindoline-1,3-dione 30 d (0.053 g, 0.11 mmol), hydrazine monohydrate (37 μL), ethanol (1.1 mL). Reverse phase HPLC of the crude product using methanol/water (1/1) as the mobile phase provided the title compound as a bright yellow semi-solid (0.006 g, 0.02 mmol, 16.0%). ¹H NMR (400 MHZ, CDCl₃) δ (ppm): 8.91 (dd, J=1.6, 4.1 Hz, 1H), 8.49 (d, J=5.8 Hz, 1H), 8.20 (dd, J=5.8, 9.3 Hz, 1H), 8.13 (dd, J=1.5, 8.3 Hz, 1H), 7.51-7.47 (m, 2H), 7.42-7.38 (m, 1H), 7.38-7.34 (m, 2H), 7.27-7.25 (m, 1H), 7.20-7.15 (m, 1H), 5.04 (s, 2H), 3.48 (t, J=5.6 Hz, 2H), 2.85 (t, J=5.6 Hz, 2H), 1.83-1.81 (m, 4H).

### CXCR4 Binding Assay

The CXCL12^{AF647} binding assay with Jurkat cells has been described previously [Schoofs et al. JoVE 2018, 133, 1-8]. Briefly, Jurkat cells were resuspended in assay buffer [Hank's Balanced Salt Solution (HBSS, Thermo Fisher Scientific), 20 mM HEPES buffer, 0.2% bovine serum albumin (Sigma-Aldrich), pH 7.4] at 3×10⁵ cells per sample and then treated with various concentrations of the compound at room temperature for 15 minutes. Afterwards, the cells were incubated with 2.9 nM CXCL12^{AF647} (in assay buffer) at room temperature for 30 minutes in the dark. Cells were fixed in 1% paraformaldehyde in DPBS and specific CXCL12^{AF647} binding [i.e. mean fluorescence intensity (MFI)] was quantified by flow cytometry (FACSArray™; Becton Dickinson). Data were analyzed with FlowJo® Software. The 50% inhibitory concentration (IC₅₀) was calculated for each compound relative to the negative (i.e. autofluorescence of untreated and unlabeled cells) and positive (i.e. untreated cells exposed to CXCL12^{AF647} only) control.

### CXCR4 Calcium Mobilization Assay

The calcium mobilization assay has been described in detail previously [Claes et al. JoVE 2018, 132, 1-9]. U87.CD4.CXCR4 cells (2×104 cells per well in DMEM/10% FBS/0.01M HEPES) were seeded in gelatin-coated (Sigma-Aldrich; 0.1% gelatin in DPBS) black-walled 96-well plates and incubated overnight at 37° ° C. and 5% CO₂. The next day, cells were loaded with the fluorescent calcium indicator Fluo-2 acetoxymethyl (AM) ester (4 μM; Abcam) and incubated at room temperature in the dark for 45 minutes. Then, cells were incubated with various concentrations of the compounds for 10 minutes prior to addition of 6.25 nM CXCL12 (in assay buffer). Fluctuations in intracellular calcium levels were measured in real time by the FLIPR Tetra® (Molecular Devices, Sunnyvale, CA, USA) in all 96 wells simultaneously. The response over baseline (after CXCL12 addition) was calculated with the ScreenWorks 4.0® software (Molecular Devices) by dividing the obtained relative light units (RLUs) through the base line measured just before CXCL12 addition. From this the IC50 value for each compound was determined taking into account the negative (i.e. untreated cells without CXCL12 stimulation) and positive (i.e. untreated cells with CXCL12 addition) control samples.

### Anti-HIV Assays

The anti-HIV assays in MT-4 cells been described in detail before [Hout et al. cited above; Vermeire et al. AIDS 2004, 18, 2115-2125]. Briefly, compound-treated MT-4 cells (5×10⁴ cells per sample in cell culture medium; 30 min incubation at 37° C.) were infected with HIV-1 (NL4-3) and HIV-2 (ROD) virus stocks. After five days, the cytopathic effect was checked microscopically and cell viability (i.e. viral replication) was evaluated using the MTS/PES-based CellTiter 96 Aqueous One Solution Cell Proliferation assay (Promega, Fitchburg, WI, USA). Absorbance was recorded using the VersaMax ELISA™ microplate reader (Molecular Devices) and analyzed with the Softmax Pro® software (Molecular Devices).

1. A compound with formula I 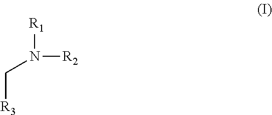
wherein R₁ is 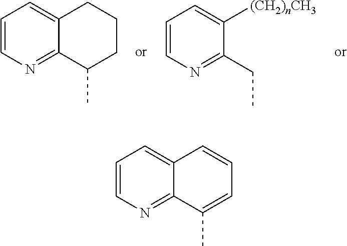
With n=0-5
wherein R₂ is —(CH₂)ₘ—NH₂ with m=1-5, and
wherein R³ is 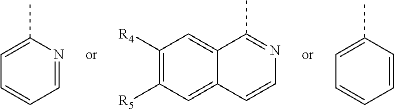
wherein R₄ and R₅ are Independently selected from H, OH, CH₃, OCH₃, OCH₂CH₃ and halogen.

2. The compound according to claim 1,
wherein R₁ is 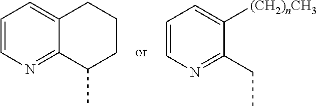
With n=0-5
wherein R₂ is —(CH₂)ₘ—NH₂ with m=1-5, and
wherein R₃ is 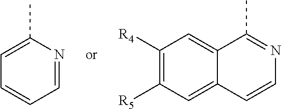
wherein R₄ and R₅ are independently selected from H, OH, CH₃, OCH₃, OCH₂CH₃ and halogen.

3. The compound according to claim 1 or 2, wherein m=4.

4. The compound according to any one of claims 1 to 3, wherein R₄ is hydrogen and R₅ is an halogen.

5. The compound according to any one of claims 1 to 4, wherein the halogen is Br, F or I.

6. The compound according to any one of claims 1 to 5, wherein n in R₁ is 1.

7. The compound according to any one of claims 1 to 6, wherein R₄ and/or R₅ is OCH₃.

8. A compound according to any one of claims 1 to 7, for use as a medicament.

9. The compound according to any one of claims 1 to 7, for use in the treatment or prevention of a viral infection.

10. The compound for use according to claim 9, wherein the viral infection is a lentiviral or retroviral infection

11. The compound for use according to claim 9 or 10, wherein the viral infection is an HIV infection.

12. A pharmaceutical composition comprising a compound according to any one of claims 1 to 7, and a pharmaceutically acceptable carrier.
